(19) United States
(12) Patent Application Publication
(10) Pub. No.: US 2026/0159570 A1
(43) Pub. Date: Jun. 11, 2026

(54) CHIMERIC CYTOKINE RECEPTOR

(71) Applicant: AUTOLUS LIMITED, London (GB)

(72) Inventors: Matteo Righi, London (GB); Shaun Cordoba, London (GB); Martin Pulé, London (GB)

(21) Appl. No.: 18/704,231

(22) PCT Filed: Oct. 24, 2022
(86) PCT No.: PCT/GB2022/052707

§ 371 Date: Apr. 24, 2024

(30) Foreign Application Priority Data
Oct. 25, 2021 (GB) 2115329.1

(51) Int. Cl. C07K 14/715; A61K 9/00; A61K 35/17; A61K 40/11; A61K 40/31; A61P 35/00; C07K 14/705; C12N 15/62; C12N 15/85

(52) U.S. Cl. CPC C07K 14/7155; A61K 9/0019; A61K 35/17; A61K 40/11; A61K 40/31; A61P 35/00; C07K 14/70503; C12N 15/62; C12N 15/85; C07K 2319/03; C07K 2319/70

(57) ABSTRACT

The present invention provides a chimeric cytokine receptor (CCR) which comprises two polypeptides: (i) a first polypeptide which comprises: (a) an ectodomain which comprises a first dimerization domain; (b) an interleukin-18 receptor 1 (IL18R1) endodomain; and (ii) a second polypeptide which comprises: (a) an ectodomain which comprises a second dimerization domain; (b) an interleukin-18 receptor accessory protein (IL18RAP) endodomain. The first polypeptide and the second polypeptide spontaneously dimerise causing IL18-type signalling to occurs through the IL18R1 and IL18RAP endodomains. The invention also provides a cell comprising such a CCR and optionally a chimeric antigen receptor (CAR) and the use of such a cell in the treatment of e.g. cancer or an autoimmune disease.

### FIELD OF THE INVENTION

The present invention relates to a chimeric cytokine receptor having an IL-18 receptor endodomain

### BACKGROUND TO THE INVENTION

Chimeric antigen receptors (CARs)

A number of immunotherapeutic agents have been described for use in cancer treatment, including therapeutic monoclonal antibodies (mAbs), bi-specific T-cell engagers and chimeric antigen receptors (CARs).

Chimeric antigen receptors are proteins which graft the specificity of a monoclonal antibody (mAb) to the effector function of a T-cell. Their usual form is that of a type I transmembrane domain protein with an antigen recognizing amino terminus, a spacer, a transmembrane domain all connected to a compound endodomain which transmits T-cell survival and activation signals.

The most common form of these molecules are fusions of single-chain variable fragments (scFv) derived from monoclonal antibodies which recognize a target antigen, fused via a spacer and a trans-membrane domain to a signaling endodomain. Such molecules result in activation of the T-cell in response to recognition by the scFv of its target. When T cells express such a CAR, they recognize and kill target cells that express the target antigen. Several CARs have been developed against tumour associated antigens, and adoptive transfer approaches using such CAR-expressing T cells are currently in clinical trial for the treatment of various cancers.

To date, the success of the CAR T cell has largely been in hematological malignancies. A CAR targeted to the B cell antigen CD19 was first used successfully to treat chronic lymphoblastic leukemia (CLL). In August 2017, the FDA approved the use of CART19 (Kymriah) to treat pediatric relapsed or refractory acute lymphoblastic leukemia (ALL) and in October of the same year, another CD19-targeting CAR (Yescarta) was approved by the FDA for adult relapsed or refractory large B cell lymphoma. Additionally, the European Medicines Agency (EMA) also approved the use of both these drugs in June of 2018. However, despite extensive research, CAR T cell therapy for solid tumours has not been nearly as successful.

One of the key hurdles which need to be overcome for a successful cell-based immunotherapeutic treatment of solid cancers is the tumour microenvironment (TME), which has been extensively characterized as hostile for T cells. CAR T-cells may fail to engraft and expand within the TME.

CAR T-cell persistence and activity can be enhanced by administration of cytokines, or by the CAR T-cells producing cytokines constitutively. However, these approaches have limitations: systemic administration of cytokines can be toxic; constitutive production of cytokines may lead to uncontrolled proliferation and transformation (Nagarkatti et al (1994) PNAS 91:7638-7642; Hassuneh et al (1997) Blood 89:610-620).

### Chimeric Cytokine Receptors (CCRs)

WO2017/029512 describes constitutively active “chimeric cytokine receptors” (CCRs) which have a Fab-like extracellular domain and the endodomain of the IL2 or IL7 receptor. The co-expression of an IL7 CCR with a chimeric antigen receptor (CAR) has been shown to help CAR T-cells engraft and expand in vivo (Achkova et al Society for the Immunotherapy of Cancer (SITC) meeting 2019 Abstract P146).

### DESCRIPTION OF THE FIGURES

FIG. 1—Schematic diagram illustrating IL-18 chimeric cytokine receptors.

A. Tethered IL18-CCR: truncated IL18 linked to either IL18R1 or IL18RAP with a flexible linker.

B. Ligand-induced IL18-CCR: the ligand (e.g. PSA) specific scFvs are linked to IL18R1 or IL18RAP endodomains via a spacer and transmembrane domain.

C. TGFβ-IL18 switch receptor: IL18R1 or IL18RAP endodomains are fused to either TGFβR1 or TGFβR2 exodomains.

D. TGFβ-IL18 CAR enhancer: TGFβ binding activated IL18 receptor enhancing first generation CAR.

E. Constitutively active IL18-CCR: Heavy chain constant domain (CH) and light chain constant domain (CL) from an antibody linked to IL18R1 or IL18RAP endodomains.

FIG. 2A—Schematic diagram illustrating the vectors used for the in vitro study

FIG. 2B—Graphs showing killing of target cells, IFNγ and IL-2 release following co-culture of transduced T-cells with target cells.

FIG. 3—Schematic diagram illustrating the experimental outline of CT26-GD2+ immunocompetent animal model in vivo study.

FIG. 4—Graphs to show tumour volume in CT26-GD2+ immunocompetent model mice following treatment with cells expressing GD2 CAR alone or co-expressing GD2 CAR with IL2, IL7 or IL18 CCR.

FIG. 5—Graphs to show T cells in peripheral blood of CT26-GD2+ immunocompetent model mice following treatment with cells expressing GD2 CAR alone or co-expressing GD2 CAR with IL2, IL7 or IL18 CCR.

FIG. 6—Kaplan Meyer survival curve of CT26-GD2+ immunocompetent model mice following treatment with cells expressing GD2 CAR alone or co-expressing GD2 CAR with IL2, IL7 or IL18 CCR.

FIG. 7—Plot showing the results of Principal Component Analysis (PCA), calculated by analysis of the 800 gene CAR T-cell panel for activated and non-activated CAR-T cells expressing either IL7- or IL18-CCR.

FIG. 8—Volcano plot showing gene expression in activated and non-activated CAR-T cells expressing IL18-CCR. IL7 CCR was used as baseline for differential gene expression calculation.

FIG. 9—Graph showing CBA assay from 1:4 E:T ratio co-culture of CAR-T cells expressing either IL7- or IL18-CCR with SupT1 GD2+ target cells.

FIG. 10—Schematic illustration of an IL-18 CCR made using coiled coil domains from an Acid/Base Leucine zipper. One polypeptide of the CCR comprises the first pair of the alpha-helices coiled coil (coiled coil 1, e.g. Acid Zipper) and the other comprises the second pair (coiled coil 2, e.g. Base Zipper). These domains spontaneously dimerise, bringing together the IL18R1 and IL18RAP endodomains, giving constitutive IL-18 signalling.

FIG. 11—Schematic illustration of an IL-18 CCR made using coiled coil domains from a SNAP-25/SNARE heterotetrametric complex. In this system, one polypeptide of the CCR comprises Chain A; one polypeptide comprises Chain B; one polypeptide comprises Chain C; and one polypeptide comprises Chain D of the SNAP-25/SNARE heterotetrametric complex. These domains spontaneously hetero-dimerise, bringing together two copies of the IL18R1 and IL18RAP endodomains, giving constitutive IL-18 signalling.

### SUMMARY OF ASPECTS OF THE INVENTION

The present inventors have surprisingly found that cells co-expressing a CAR and a CCR having an IL-18 receptor endodomain show improved CAR-T cell persistence and anti-tumour activity in vivo in an immunocompetent animal model than equivalent cells co-expressing a CAR and an IL-7 or IL-2 CCR.

Thus, in a first aspect, the invention provides a chimeric cytokine receptor (CCR) which comprises two polypeptides:
(i) a first polypeptide which comprises:
   (a) an ectodomain which comprises a first dimerization domain;
   (b) an interleukin-18 receptor 1 (IL18R1) endodomain; and
(ii) a second polypeptide which comprises:
   (a) an ectodomain which comprises a second dimerization domain which spontaneously dimerises with the first dimerization domain;
   (b) an interleukin-18 receptor accessory protein (IL18RAP) endodomain.

The dimerization domain may be based on the dimerization domain of an antibody. In this respect, one of the first and second dimerization domains may comprise a heavy chain constant domain (CH) and the other may comprise a light chain constant domain (CL).

Alternatively, the first and second dimerization domains may comprise coiled-coil domains.

For example, the first and second dimerization domains may comprise leucine zipper domains, such as Fos/Jun domains or docking domain (DDD1)/anchoring domain (AD1) leucine zipper domains.

The CCR may be a tetramer. In this embodiment the CCR comprises four polypeptides:
(i) a first polypeptide which comprises:
   (a) an ectodomain which comprises a first tetramerization domain;
   (b) an interleukin-18 receptor 1 (IL18R1) endodomain;
(ii) a second polypeptide which comprises:
   (a) an ectodomain which comprises a second tetramerization domain;
   (b) an interleukin-18 receptor accessory protein (IL18RAP) endodomain;
(i) a third polypeptide which comprises:
   (a) an ectodomain which comprises a third tetramerization domain;
   (b) an interleukin-18 receptor 1 (IL18R1) endodomain;
(ii) a fourth polypeptide which comprises:
   (a) an ectodomain which comprises a fourth tetramerization domain;
   (b) an interleukin-18 receptor accessory protein (IL18RAP) endodomain.

In this embodiment, the tetramerization domains may, for example, comprise chains A, B, C and D of the SNAP-25/SNARE heterotetrametric complex.

In a second aspect, the invention provides a cell which comprises a chimeric cytokine receptor according to the first aspect of the invention.

The cell may also comprise a chimeric antigen receptor.

In a third aspect, there is provided a nucleic acid construct encoding a chimeric cytokine receptor (CCR) according to the first aspect of the invention, which nucleic acid construct comprises a first nucleic acid sequence encoding the first polypeptide and a second nucleic acid sequence encoding the second polypeptide.

In order to produce a tetrameric CCR, the nucleic acid construct may comprise a first nucleic acid sequence encoding the first polypeptide; a second nucleic acid sequence encoding the second polypeptide; a third nucleic acid sequence encoding the third polypeptide; and a fourth nucleic acid sequence encoding the fourth polypeptide.

The nucleic acid construct may also comprise a nucleic acid sequence which encodes a chimeric antigen receptor.

In a fourth aspect there is provided a vector comprising a nucleic acid construct according to the third aspect of the invention.

In a fifth aspect, these is provided a kit which comprises:
i) a vector comprising a nucleic acid sequence encoding a first polypeptide as defined above; and
ii) a vector comprising a nucleic acid sequence encoding a second polypeptide as defined above.

In order to produce a tetrameric CCR, the kit may comprise
i) a vector comprising a nucleic acid sequence encoding a first polypeptide as defined above;
ii) a vector comprising a nucleic acid sequence encoding a second polypeptide as defined above;
iii) a vector comprising a nucleic acid sequence encoding a third polypeptide as defined above; and
iv) a vector comprising a nucleic acid sequence encoding a fourth polypeptide as defined above.

In a sixth aspect, there is provided a method for making a cell according to the second aspect of the invention, which comprises the step of introducing: a nucleic acid construct according to the third aspect of the invention; a vector according to the fourth aspect of the invention; or a kit of vectors according to the fifth aspect of the invention, into a cell ex vivo.

In a seventh aspect, there is provided a pharmaceutical composition comprising a plurality of cells according to the second aspect of the invention.

In an eighth aspect, there is provided a pharmaceutical composition according to the seventh aspect of the invention for use in treating cancer or an autoimmune disease.

In a ninth aspect, there is provided a method for treating cancer or an autoimmune disease which comprises the step of administering a pharmaceutical composition according to the seventh aspect of the invention to a subject.

In an eleventh aspect, there is provided the use of a plurality of cells according to the second aspect of the invention for use in the manufacture of a pharmaceutical composition for the treatment of cancer or an autoimmune disease.

### FURTHER ASPECTS OF THE INVENTION

Further aspects of the invention are presented in the following numbered paragraphs:
1. A chimeric cytokine receptor (CCR) which comprises two polypeptides:
   (i) a first polypeptide which comprises:
      (a) an ectodomain which comprises a first dimerization domain;
      (b) an interleukin-18 receptor 1 (IL18R1) endodomain; and
   (ii) a second polypeptide which comprises:
      (a) an ectodomain which comprises a second dimerization domain;
      (b) an interleukin-18 receptor accessory protein (IL18RAP) endodomain.
2. A CCR according to paragraph 1, wherein the first and second dimerization domains dimerise spontaneously.
3. A CCR according to paragraph 2, wherein one of the first and second dimerization domains comprises a heavy chain constant domain (CH) and the other comprises a light chain constant domain (CL).
4. A CCR according to paragraph 1, wherein the first and second dimerization domains dimerise in the presence of a ligand.
5. A CCR according to paragraph 4, wherein the first and second dimerization domains are single-chain variable fragments (scFvs) or single domain binders (dAbs) which independently bind the ligand.
6. A CCR according to paragraph 4, wherein one of the first and second dimerization domains comprises a heavy chain variable domain (VH) and the other comprises a light chain variable domain (VL) which together bind the ligand.
7. A CCR according to paragraph 4, wherein the wherein one of the first and second dimerization domains comprises the extracellular region of a transforming growth factor beta receptor I (TBRI), and the other comprises the extracellular region of a transforming growth factor beta receptor II (TBRII) and the ligand is transforming growth factor beta (TGFβ).
8. A CCR according to paragraph 8, wherein the first polypeptide comprises:
   (i) an antigen binding domain,
   (ii) a linker,
   (iii) an extracellular region of a first transforming growth factor beta receptor (TBR),
   (iv) a first transmembrane domain,
   (v) an interleukin-18 receptor 1 (IL18R1) endodomain, and
   (vi) a CD3z endodomain;
and wherein the second polypeptide comprises:
   (i) an extracellular region of a second TBR,
   (ii) a second transmembrane domain, and
   (iii) an interleukin-18 receptor accessory protein (IL18RAP) endodomain;
or wherein the first polypeptide comprises:
   (i) an antigen binding domain,
   (ii) a linker,
   (iii) an extracellular region of a first transforming growth factor beta receptor (TBR),
   (iv) a first transmembrane domain, and
   (v) an IL18RAP endodomain, and
   (vi) a CD3z endodomain;
and wherein the second polypeptide comprises:
   (i) an extracellular region of a second TBR,
   (ii) a second transmembrane domain, and
   (iii) an IL18R1 endodomain.
9. A cell which comprises a chimeric cytokine receptor according to any preceding paragraph.
10. A cell according to paragraph 6, which also comprises a chimeric antigen receptor.
11. A nucleic acid construct encoding a chimeric cytokine receptor (CCR) according to any of paragraphs 1 to 8, which nucleic acid construct comprises a first nucleic acid sequence encoding the first polypeptide and a second nucleic acid sequence encoding the second polypeptide.
12. A vector comprising a nucleic acid construct according to paragraph 11.
13. A kit which comprises:
   i) a vector comprising a nucleic acid sequence encoding a first polypeptide as defined in any of paragraphs 1 to 8; and
   ii) a vector comprising a nucleic acid sequence encoding a second polypeptide as defined in any of paragraphs 1 to 8.
14. A method for making a cell according to paragraph 9 or 10, which comprises the step of introducing: a nucleic acid construct according to paragraph 11; a vector according to paragraph 12; or a kit of vectors according to paragraph 13, into a cell ex vivo.
15. A pharmaceutical composition comprising a plurality of cells according to paragraph 11 or 12.
16. A pharmaceutical composition according to paragraph 15 for use in treating cancer or an autoimmune disease.
17. A method for treating cancer or an autoimmune disease which comprises the step of administering a pharmaceutical composition according to claim 15 to a 25 subject.
18. The use of a plurality of cells according to claim 9 or 10 for use in the manufacture of a pharmaceutical composition for the treatment of cancer or an autoimmune disease.

### DETAILED DESCRIPTION

### Chimeric Cytokine Receptor (CCR)

A chimeric cytokine receptor (CCR) is a molecule which comprises a cytokine receptor endodomain and an exodomain. The exodomain may be derived from a protein other than a cytokine receptor. The exodomain may be derived from a protein other than IL-18 receptor.

The exodomain may make the chimeric cytokine receptor constitutively active. For example, the CCR may comprise an exodomain which dimerises spontaneously.

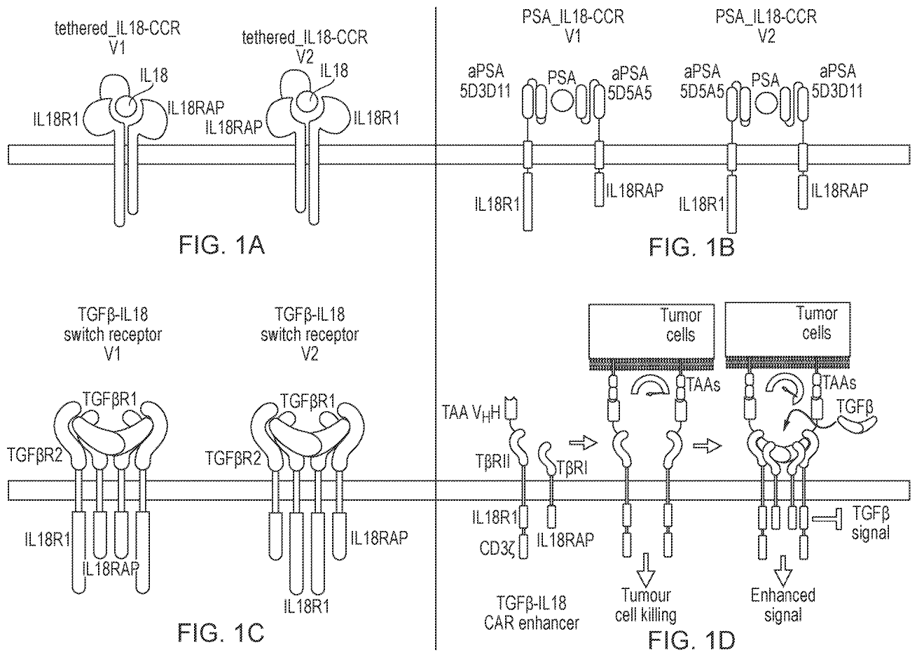

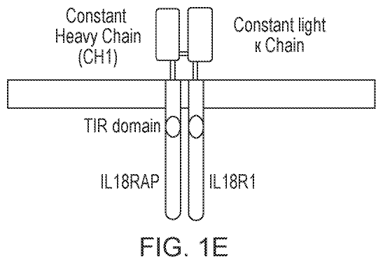

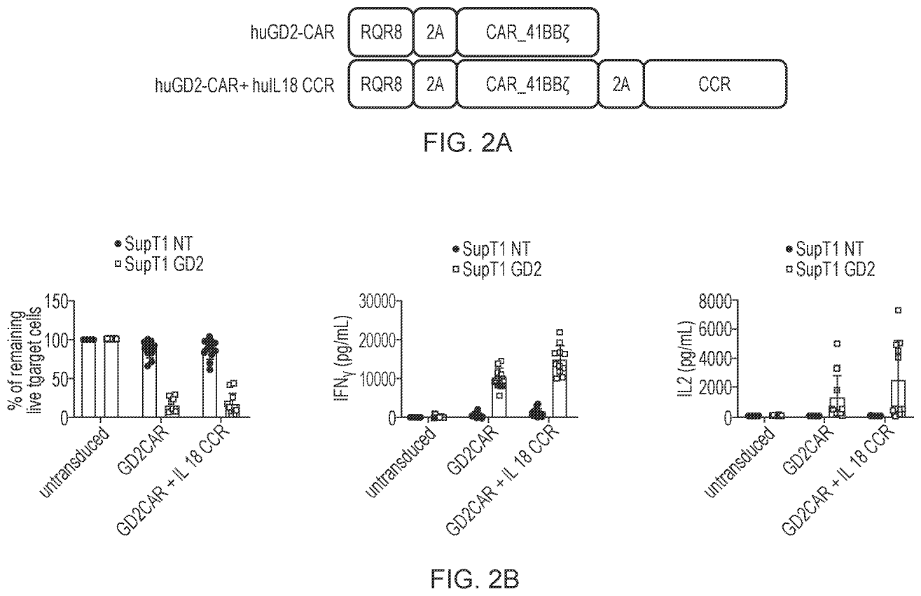

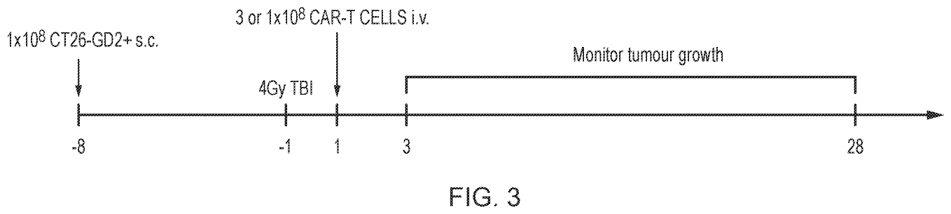

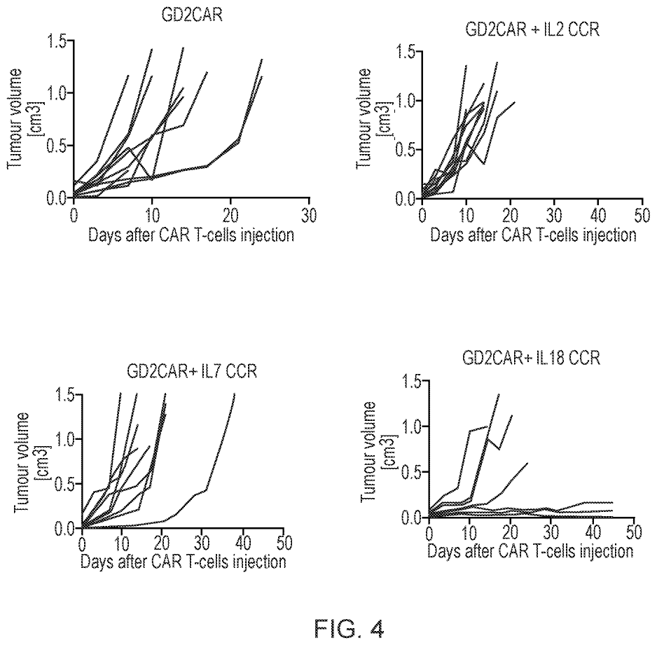

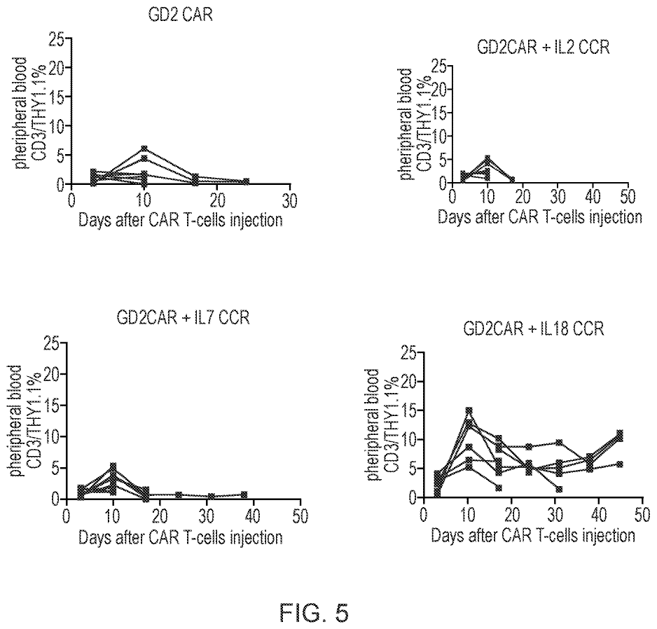

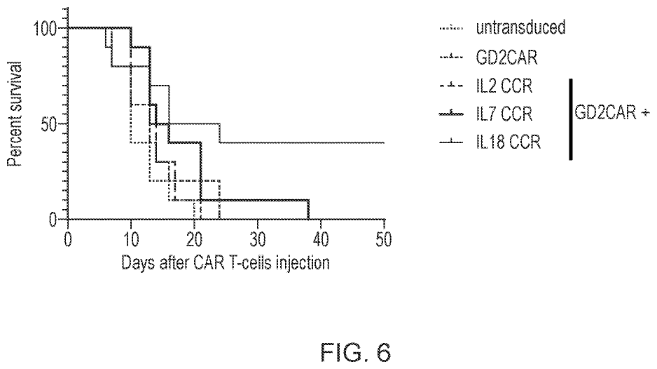

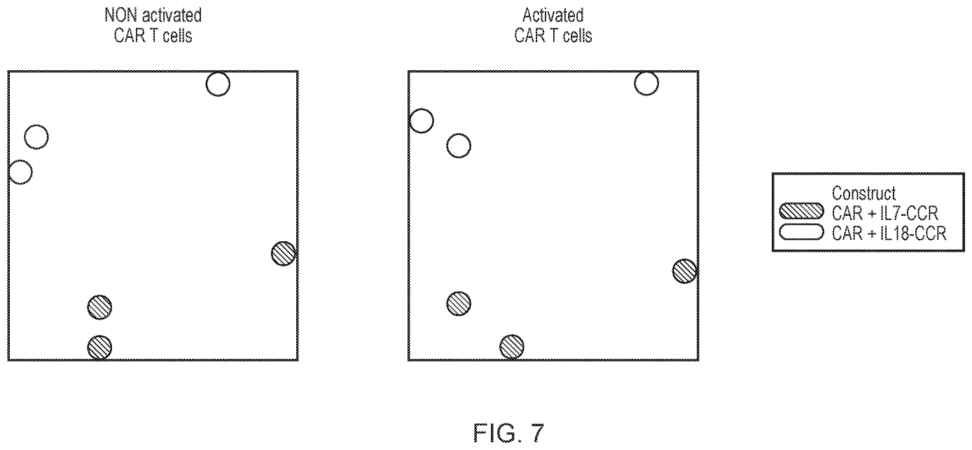

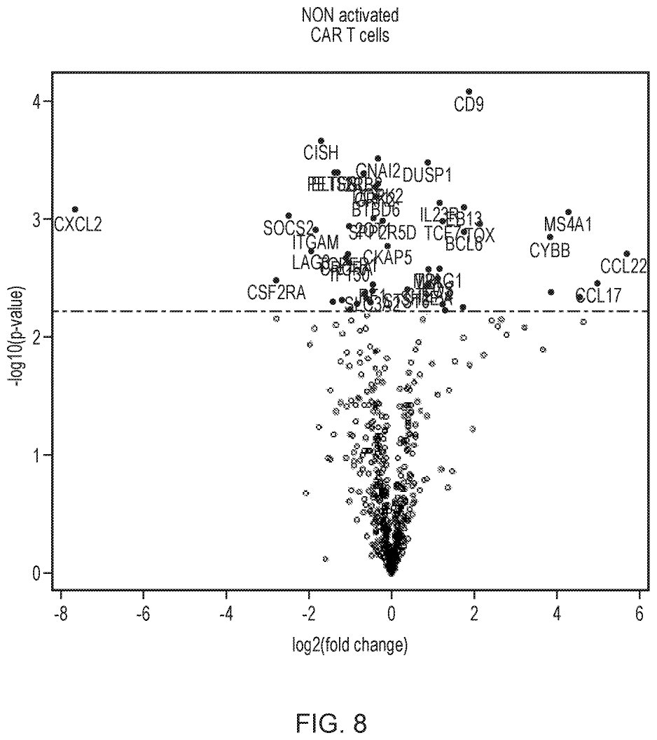

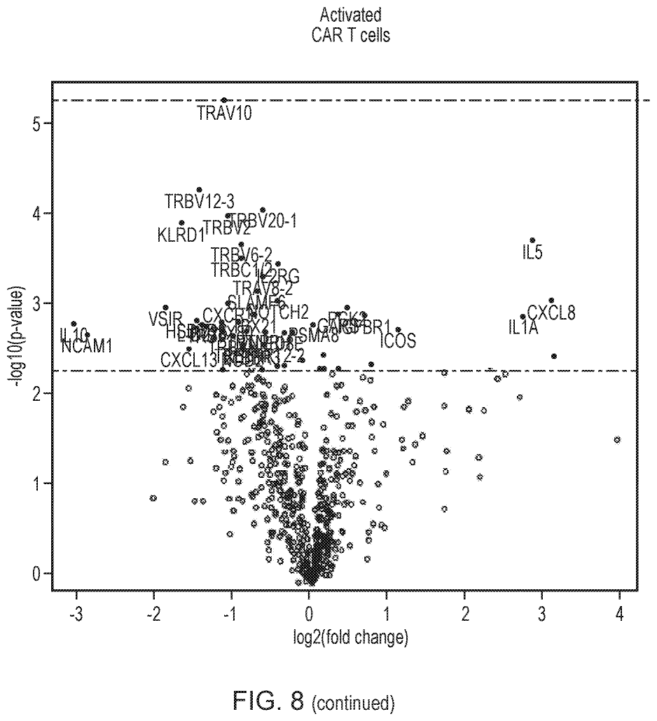

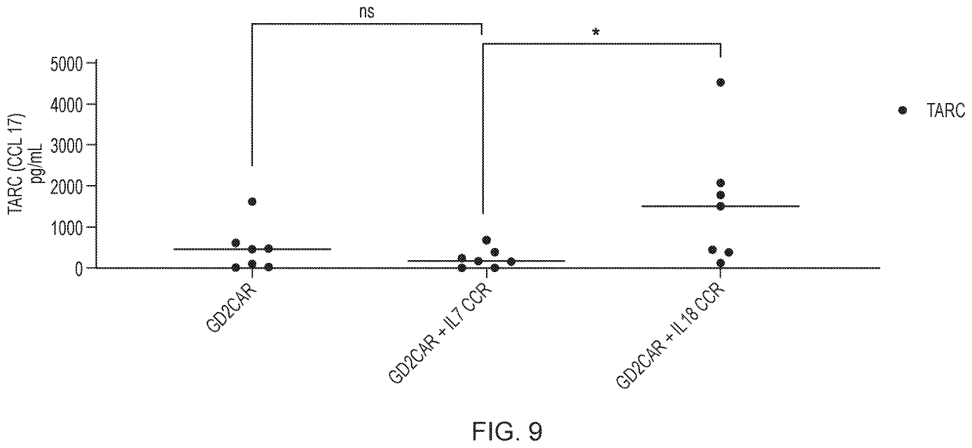

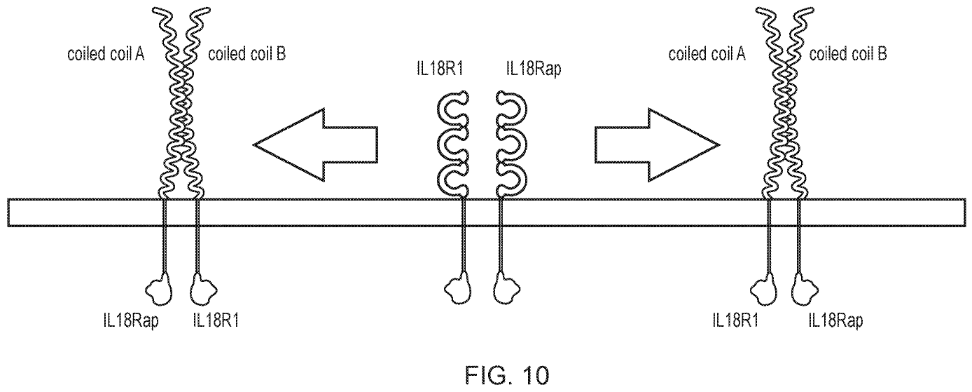

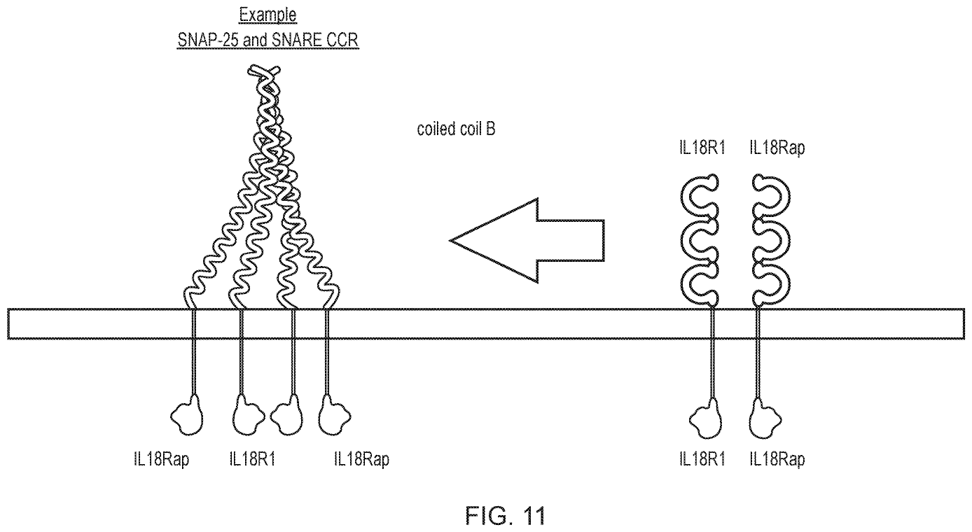

In the arrangement illustrated schematically in FIG. 1E, one polypeptide of the CCR comprises a heavy chain constant domain (CH) and the other comprises a light chain constant domain (CL). These domains spontaneously dimerise, bringing together the IL18R1 and IL18RAP endodomains, giving constitutive IL-18 signalling.

Alternatively, a constitutively active CCR or the invention may comprise a coiled coil domain giving spontaneous dimerization or multimerization (e.g. tetramerization).

In the arrangement illustrated schematically in FIG. 10, one polypeptide of the CCR comprises the first pair of the alpha-helices coiled coil (coiled coil 1, e.g. Acid Zipper) and the other comprises the second pair (coiled coil 2, e.g. Base Zipper). These domains spontaneously dimerise, bringing together the IL18R1 and IL18RAP endodomains, giving constitutive IL-18 signalling.

In the arrangement illustrated schematically in Figure FIG. 11, one polypeptide of the CCR comprises Chain A; one polypeptide comprises Chain B; one polypeptide comprises Chain C; and one polypeptide comprises Chain D of the SNAP-25/SNARE heterotetrametric complex. These domains spontaneously hetero-dimerise, bringing together two copies of the IL18R1 and IL18RAP endodomains, giving constitutive IL-18 signalling.

In the arrangement illustrated schematically in FIG. 1A, the receptor comprises IL-18R α and β chains, and one of the chains also comprises an IL-18 molecule tethered to the polypeptide by way of a linker. The tethered IL-18 binds and dimerises the IL18R1 and IL18RAP chains, due to its permanent physical proximity, giving constitutive IL-18 signalling.

Alternatively, the exodomain may make the chimeric cytokine receptor active in the presence of a chemical inducer of dimerization (CID) or ligand.

In the arrangement illustrated schematically in FIG. 1B, the first and second polypeptides of the CCR each comprise a binding domain, such as an scFv or dAb, each or which bind a ligand. In the presence of the ligand, the exodomains both bind, bringing the endodomains together and giving ligand-dependent IL-18 signalling.

The ligand may, for example, be a tumour secreted factor such as prostate-specific antigen (PSA), carcinoembryonic antigen (CEA) and vascular endothelial growth factor (VEGF) or CA125

In the arrangement illustrated schematically in FIG. 1C, the first and second polypeptides of the CCR each comprise an exodomain from a TGFβ receptor. For example, one polypeptide may comprise an exodomain from TGFβ receptor I and the other polypeptide may comprise an exodomain from TGFβ receptor II. In the presence of TGFβ, the two TGFβ receptor endodomains come together causing IL-18-type signalling.

The arrangement illustrated schematically in FIG. 1D is also IL-18-type signalling is also triggered by the presence of TGFβ. In this arrangement, one of the polypeptides comprises an ITAM-containing endodomain and an antigen-binding domain. This polypeptide acts like a CAR in that it induces activation of the cell in the presence of the target antigen. This polypeptide also comprises one of the IL-18 receptor endodomains and a TGFβ receptor exodomain. The other polypeptide in the CCR comprises a second TGFβ receptor exodomain and the other IL-18 receptor endodomain chain. In the presence of TGFβ, the two TGFβ receptor endodomains come together causing IL-18-type signalling. In the presence of both TGFβ and target antigen, the cell receives both an activation signal and an IL-18-type proliferation signal.

### IL18 Receptor Endodomain

IL-18 belongs to the IL-1 superfamily and is produced mainly by macrophages but also other cell types, and has pleiotropic functions in cell stimulation. IL-18 is a proinflammatory cytokine that facilitates type 1 responses. Together with IL-12, it induces cell-mediated immunity following infection with microbial products like lipopolysaccharide (LPS). IL-18 in combination with IL12 acts on CD4, CD8 T cells and NK cells to induce IFNγ production, type II interferon that plays an important role in activating the macrophages or other cells. The combination of this IL-18 and IL-12 has been shown to inhibit IL-4 dependent IgE and IgG1 production and enhance IgG2a production in B cells. In vivo it has been reported that, without IL-12 or IL-15, IL-18 does not induce IFNγ production, but plays an important role in the differentiation of naive T cells into Th2 cells and stimulates mast cells and basophils to produce IL-4, IL-13, and chemical mediators such as histamine.

The IL-18 receptor consists of the inducible component an interleukin-18 receptor 1 (IL18R1), which binds the mature IL-18 with low affinity and the constitutively expressed co-receptor interleukin-18 receptor accessory protein (IL18RAP). IL-18 binds the ligand receptor IL18R1, inducing the recruitment of IL18RAP to form a high affinity complex, which signals through the toll/interleukin-1 receptor (TIR) domain. This signaling domain recruits MyD88 adaptor protein that activates proinflammatory programs and NF-κB pathway.

The amino acid sequence for human IL18 receptor 1 (also known as IL18 receptor α chain) is available from Uniprot Accession No. Q13478 and shown below as SEQ ID No. 1. In this sequence, residues 22-329 represent the extracellular domain, residues 330-350 represent the transmembrane domain and residues 351-541 represent the cytoplasmic domain.

-human IL18R1
SEQ ID No. 1
MNCRELPLTLWVLISVSTAESCTSRPHITVVEGEPFYLKHCSCSLAHEI
ETTTKSWYKSSGSQEHVELNPRSSSRIALHDCVLEFWPVELNDTGSYFF
QMKNYTQKWKLNVIRRNKHSCFTERQVTSKIVEVKKFFQITCENSYYQT
LVNSTSLYKNCKKLLLENNKNPTIKKNAEFEDQGYYSCVHFLHHNGKLF
NITKTFNITIVEDRSNIVPVLLGPKLNHVAVELGKNVRLNCSALLNEED
VIYWMFGEENGSDPNIHEEKEMRIMTPEGKWHASKVLRIENIGESNLNV
LYNCTVASTGGTDTKSFILVRKADMADIPGHVFTRGMIIAVLILVAVVC
LVTVCVIYRVDLVLFYRHLTRRDETLTDGKTYDAFVSYLKECRPENGEE
HTFAVEILPRVLEKHFGYKLCIFERDVVPGGAVVDEIHSLIEKSRRLII
VLSKSYMSNEVRYELESGLHEALVERKIKIILIEFTPVTDFTFLPQSLK
LLKSHRVLKWKADKSLSYNSRFWKNLLYLMPAKTVKPGRDEPEVLPVLS
ES

For embodiments of the invention in which one polypeptide comprises IL18R1 exodomain and endodomain, for example, the embodiment illustrated in FIG. 1A, the polypeptide may comprise residues 22 to 541 of SEQ ID No. 1.

For embodiments of the invention in which one polypeptide comprises the IL18R1 endodomain and a heterologous exodomain, the polypeptide may comprise residues 330-541 of SEQ ID No. 1 (if the polypeptide includes the IL18R1 transmembrane domain—SEQ ID No. 8 and 9 below) or residues 351-541 (if the polypeptide has a heterologous transmembrane domain).

The CCR of the invention may comprise a variant of part or all of the sequence shown as SEQ ID No.1, which variant may for example have 70, 80, 90, 95 or 99% identity to the sequence shown as SEQ ID No. 1 (or the identified portion thereof), provided that a polypeptide comprising the variant sequence retains the capacity to trigger an IL-18 signal in a cell when dimerised with a polypeptide comprising an IL18RAP endodomain.

The amino acid sequence for human IL18 receptor accessory protein (IL18RAP, also known as IL18 receptor β chain) is available from Uniprot Accession No. 095256 and shown below as SEQ ID No. 2. In this sequence, residues 20-356 represent the extracellular domain, residues 357-377 represent the transmembrane domain and residues 378-599 represent the cytoplasmic domain.

-human IL18RAP
SEQ ID No. 2
MLCLGWIFLWLVAGERIKGFNISGCSTKKLLWTYSTRSEEEFVLFCDLP
EPQKSHFCHRNRLSPKQVPEHLPFMGSNDLSDVQWYQQPSNGDPLEDIR
KSYPHIIQDKCTLHFLTPGVNNSGSYICRPKMIKSPYDVACCVKMILEV
KPQTNASCEYSASHKQDLLLGSTGSISCPSLSCQSDAQSPAVTWYKNGK
LLSVERSNRIVVDEVYDYHQGTYVCDYTQSDTVSSWTVRAVVQVRTIVG
DTKLKPDILDPVEDTLEVELGKPLTISCKARFGFERVFNPVIKWYIKDS
DLEWEVSVPEAKSIKSTLKDEIIERNIILEKVTQRDLRRKFVCFVQNSI
GNTTQSVQLKEKRGVVLLYILLGTIGTLVAVLAASALLYRHWIEIVLLY
RTYQSKDQTLGDKKDFDAFVSYAKWSSFPSEATSSLSEEHLALSLFPDV
LENKYGYSLCLLERDVAPGGVYAEDIVSIIKRSRRGIFILSPNYVNGPS
IFELQAAVNLALDDQTLKLILIKFCYFQEPESLPHLVKKALRVLPTVTW
RGLKSVPPNSRFWAKMRYHMPVKNSQGFTWNQLRITSRIFQWKGLSRTE
TTGRSSQPKEW

For embodiments of the invention in which one polypeptide comprises IL18RAP exodomain and endodomain; for example, the embodiment illustrated in FIG. 1A, the polypeptide may comprise residues 20 to 599 of SEQ ID No. 2.

For embodiments of the invention in which one polypeptide comprises the IL18RAP endodomain and a heterologous exodomain; for example the embodiments illustrated in FIG. 1B-E; the polypeptide may comprise residues 357-599 of SEQ ID No. 2 (if the polypeptide includes the IL18RAP transmembrane domain—see SEQ ID No. 12 and 13 below) or residues 378-599 (if the polypeptide has a heterologous transmembrane domain).

The CCR of the invention may comprise a variant of part or all of the sequence shown as SEQ ID No.2, which variant may for example have 70, 80, 90, 95 or 99% identity to the sequence shown as SEQ ID No. 2 (or the identified portion thereof), provided that a polypeptide comprising the variant sequence retains the capacity to trigger an IL-18 signal in a cell when dimerised with a polypeptide comprising an IL18R1 endodomain.

### Truncations of the IL18 Endodomains

It is known that truncation of some type I cytokine receptor endodomains can increase proliferation when the CCR is expressed in a T-cell with regard to the wild-type version. WO2021/023987 describes a study in which the IL-2 receptor β-chain of a constitutively active CCR was given successive 20mer deletions. The ccrs were expressed in T cells and the absolute number of viable, transduced cells was assessed by flow cytometry. Truncation of the IL-2 receptor β-chain endodomain by 20 or 40 amino acids (i.e. from amino acids 266-551 to 266-531 and 266-511 respectively) increased proliferation, with the highest level of proliferation observed for the IL2Rbeta aa266-511. Further truncation of the IL-2 receptor β-chain endodomain results in a step-wise reduction in proliferation from aa266-471>aa266-451>aa 266-411>aa266-391>aa266-371, at which point it plateaued with further deletions having no significant effect of the level of proliferation. It is therefore possible to reduce the activity of chimeric cytokine receptors by truncation of one or both cytokine receptor endodomains. It is also possible to “tailor” CCRs to have a desired level of cytokine production by selecting an endodomain truncation which gives the desired level of activity.

A truncated version of IL18R1 or IL18 RAP may have a truncation of between 10 and 180 amino acids, for example, between 20 and 170 amino acids, between 40 and 160, between 60 and 150, between 80 and 130 or between 100 and 120 amino acids from the C terminus. The truncation may be involve removal of between 1 and 40 amino acids from the C terminus, for example between 5 and 35, 10 and 30, or 15 and 25 amino acids from the C terminus.

Table 1 shows truncations of the IL18R1 and IL18RAP endodomain sequences. The CCR or the invention may comprise one of the truncated versions of IL18R1 and/or IL18RAP shown in Table 1. A truncated version of IL18R1 and/or IL18RAP may have one of the sequences shown as SEQ ID No. 36 to 53, and 62. A truncated version of IL18R1 and/or IL18RAP may have a sequence “between” two of the truncated sequences shown in Table 1, for example, a sequence “between IL18R1 truncation 10 (SEQ ID NO. 44) which is missing the last 11 amino acids of the endodomain sequence; and IL18R1 truncation 9 (SEQ ID NO. 43) which is missing the last 31 amino acids of the endodomain sequence, are truncations of the last 30, 29, 28 . . . etc . . . 14, 13 and 12 amino acids of the IL18R1 endodomain sequence.

The truncation may be such that a CCR made with the truncated version of the IL18R1 or IL18RAP endodomain, when expressed in a T-cell increases proliferation of the T cell compared to an equivalent cell expressing an equivalent CCR having the wild-type (i.e. untruncated) endodomain sequence.

TABLE 1
IL18R1 and IL18RAP truncations

| truncations | endodomain aminoacidic sequence |
|---|---|
| IL18R1 full | YRVDLVLFYRHLTRRDETLTDGKTYDAFVSYLKECRPENGEEHTFAVEILPRV |
| length | LEKHFGYKLCIFERDVVPGGAVVDEIHSLIEKSRRLIIVLSKSYMSNEVRYELES |
| | GLHEALVERKIKIILIEFTPVTDFTFLPQSLKLLKSHRVLKWKADKSLSYNSRFW |
| | KNLLYLMPAKTVKPGRDEPEVLPVLSES (SEQ ID No. 9) |
| IL18R1 | YR |
| Truncation 1 | |
| IL18R1 | YRVDLVLFYRHLTRRDETLT (SEQ ID No. 36) |
| truncation 2 | |
| IL18R1 | YRVDLVLFYRHLTRRDETLTDGKTYDAFVSYLKECRPENG (SEQ ID No. 37) |
| truncation 3 | |
| IL18R1 | YRVDLVLFYRHLTRRDETLTDGKTYDAFVSYLKECRPENGEEHTFAVEILPRV |
| truncation 4 | LEKHFGY (SEQ ID No. 38) |
| IL18R1 | YRVDLVLFYRHLTRRDETLTDGKTYDAFVSYLKECRPENGEEHTFAVEILPRV |
| truncation 5 | LEKHFGYKLCIFERDVVPGGAVVDEIH (SEQ ID No. 39) |
| IL18R1 | YRVDLVLFYRHLTRRDETLTDGKTYDAFVSYLKECRPENGEEHTFAVEILPRV |
| truncation 6 | LEKHFGYKLCIFERDVVPGGAVVDEIHSLIEKSRRLIIVLSKSYMSN (SEQ ID |
| | No. 40) |
| IL18R1 | YRVDLVLFYRHLTRRDETLTDGKTYDAFVSYLKECRPENGEEHTFAVEILPRV |
| truncation 7 | LEKHFGYKLCIFERDVVPGGAVVDEIHSLIEKSRRLIIVLSKSYMSNEVRYELES |
| | GLHEALVERKIK (SEQ ID No. 41) |
| IL18R1 | YRVDLVLFYRHLTRRDETLTDGKTYDAFVSYLKECRPENGEEHTFAVEILPRV |
| truncation 8 | LEKHFGYKLCIFERDVVPGGAVVDEIHSLIEKSRRLIIVLSKSYMSNEVRYELES |
| | GLHEALVERKIKIILIEFTPVTDFTFLPQSLK (SEQ ID No. 42) |
| IL18R1 | YRVDLVLFYRHLTRRDETLTDGKTYDAFVSYLKECRPENGEEHTFAVEILPRV |
| truncation 9 | LEKHFGYKLCIFERDVVPGGAVVDEIHSLIEKSRRLIIVLSKSYMSNEVRYELES |
| | GLHEALVERKIKIILIEFTPVTDFTFLPQSLKLLKSHRVLKWKADKSLSYNS |
| | (SEQ ID No. 43) |
| IL18R1 | YRVDLVLFYRHLTRRDETLTDGKTYDAFVSYLKECRPENGEEHTFAVEILPRV |
| truncation | LEKHFGYKLCIFERDVVPGGAVVDEIHSLIEKSRRLIIVLSKSYMSNEVRYELES |
| 10 | GLHEALVERKIKIILIEFTPVTDFTFLPQSLKLLKSHRVLKWKADKSLSYNSRFW |
| | KNLLYLMPAKTVKPGRD (SEQ ID No. 44) |
| IL18RAP full | SALLYRHWIEIVLLYRTYQSKDQTLGDKKDFDAFVSYAKWSSFPSEATSSLSE |
| length | EHLALSLFPDVLENKYGYSLCLLERDVAPGGVYAEDIVSIIKRSRRGIFILSPNY |
| | VNGPSIFELQAAVNLALDDQTLKLILIKFCYFQEPESLPHLVKKALRVLPTVTW |
| | RGLKSVPPNSRFWAKMRYHMPVKNSQGFTWNQLRITSRIFQWKGLSRTETT |
| | GRSSQPK (SEQ ID No. 13) |
| IL18RAP | SA |
| truncation 1 | |
| IL18RAP | SALLYRHWIEIVLLYRTYQS (SEQ ID No. 45) |
| truncation 2 | |
| IL18RAP | SALLYRHWIEIVLLYRTYQSKDQTLGDKKDFDAFVSYAKW (SEQ ID No. 46) |
| truncation 3 | |
| IL18RAP | SALLYRHWIEIVLLYRTYQSKDQTLGDKKDFDAFVSYAKWSSFPSEATSSLSE |
| truncation 4 | EHLALSL (SEQ ID No. 47) |
| IL18RAP | SALLYRHWIEIVLLYRTYQSKDQTLGDKKDFDAFVSYAKWSSFPSEATSSLSE |
| truncation 5 | EHLALSLFPDVLENKYGYSLCLLERDV (SEQ ID No. 48) |
| IL18RAP | SALLYRHWIEIVLLYRTYQSKDQTLGDKKDFDAFVSYAKWSSFPSEATSSLSE |
| truncation 6 | EHLALSLFPDVLENKYGYSLCLLERDVAPGGVYAEDIVSIIKRSRRG (SEQ ID |
| | No. 49) |
| IL18RAP | SALLYRHWIEIVLLYRTYQSKDQTLGDKKDFDAFVSYAKWSSFPSEATSSLSE |
| truncation 7 | EHLALSLFPDVLENKYGYSLCLLERDVAPGGVYAEDIVSIIKRSRRGIFILSPNY |
| | VNGPSIFELQAA (SEQ ID No. 50) |
| IL18RAP | SALLYRHWIEIVLLYRTYQSKDQTLGDKKDFDAFVSYAKWSSFPSEATSSLSE |
| truncation 8 | EHLALSLFPDVLENKYGYSLCLLERDVAPGGVYAEDIVSIIKRSRRGIFILSPNY |
| | VNGPSIFELQAAVNLALDDQTLKLILIKFCYF (SEQ ID No. 51) |
| IL18RAP | SALLYRHWIEIVLLYRTYQSKDQTLGDKKDFDAFVSYAKWSSFPSEATSSLSE |
| truncation 9 | EHLALSLFPDVLENKYGYSLCLLERDVAPGGVYAEDIVSIIKRSRRGIFILSPNY |
| | VNGPSIFELQAAVNLALDDQTLKLILIKFCYFQEPESLPHLVKKALRVLPTV |
| | (SEQ ID No. 52) |
| IL18RAP | SALLYRHWIEIVLLYRTYQSKDQTLGDKKDFDAFVSYAKWSSFPSEATSSLSE |
| truncation | EHLALSLFPDVLENKYGYSLCLLERDVAPGGVYAEDIVSIIKRSRRGIFILSPNY |
| 10 | VNGPSIFELQAAVNLALDDQTLKLILIKFCYFQEPESLPHLVKKALRVLPTVTW |
| | RGLKSVPPNSRFWAKMRY (SEQ ID No. 62) |
| IL18RAP | SALLYRHWIEIVLLYRTYQSKDQTLGDKKDFDAFVSYAKWSSFPSEATSSLSE |
| truncation | EHLALSLFPDVLENKYGYSLCLLERDVAPGGVYAEDIVSIIKRSRRGIFILSPNY |
| 11 | VNGPSIFELQAAVNLALDDQTLKLILIKFCYFQEPESLPHLVKKALRVLPTVTW |
| | RGLKSVPPNSRFWAKMRYHMPVKNSQGFTWNQLRITSR (SEQ ID No. 53) |

### Dimerising Exodomain

The chimeric cytokine receptor of the present invention comprises first and second polypeptides which dimerise, bringing together the IL18R1 and IL18RAP endodomains.

Dimerisation may occur spontaneously, in which case the chimeric transmembrane protein will be constitutively active. Alternatively, dimerization may occur only in the presence of a chemical inducer of dimerization (CID) or ligand in which case the transmembrane protein only causes cytokine-type signalling in the presence of the CID or ligand.

### Constitutively Active Systems

Where the dimerization domain spontaneously heterodimerizes, it may be based on the dimerization domain of an antibody. In particular it may comprise the dimerization portion of a heavy chain constant domain (CH) and a light chain constant domain (CL). The “dimerization portion” of a constant domain is the part of the sequence which forms the inter-chain disulphide bond.

A suitable arrangement of such a CCR having a “Fab”-type exodomain is illustrated schematically in FIG. 1E.

The light chain constant domain may comprise the sequence shown as SEQ ID No. 3 and the heavy chain constant domain may comprise the sequence shown as SEQ ID No. 4.

(Human kappa)
SEQ ID No. 3
RTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPV
TKSFNRGEC
(Human CH1)
SEQ ID No. 4
STKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRV

An illustrative sequences for a “Fab-type” CCR as shown in FIG. 1E is shown below, with the individual components identified.

### “Fab-Type” OCR

Signal peptide
(SEQ ID No. 5)
MGWSCIILFLVATATGVHS
Light kappa
(SEQ ID No. 3)
RTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPV
TKSFNRGEC
Hinge
(SEQ ID No. 6)
EPKSCDKTHTCPPCP
Linker
(SEQ ID No. 7)
KDPK
IL18R1 transmembrane
(SEQ ID No. 8)
GMIIAVLILVAVVCLVTVCVI
IL18R1 endodomain
(SEQ ID No. 9)
YRVDLVLFYRHLTRRDETLTDGKTYDAFVSYLKECRPENGEEHTFAVEI
LPRVLEKHFGYKLCIFERDVVPGGAVVDEIHSLIEKSRRLIIVLSKSYM
SNEVRYELESGLHEALVERKIKIILIEFTPVTDFTFLPQSLKLLKSHRV
LKWKADKSLSYNSRFWKNLLYLMPAKTVKPGRDEPEVLPVLSES
T2A
(SEQ ID No. 10)
EGRGSLLTCGDVEENPGP
Signal peptide
(SEQ ID No. 11)
METDTLILWVLLLLVPGSTG
Human CH1
(SEQ ID No. 4)
STKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRV
Hinge
(SEQ ID No. 6)
EPKSCDKTHTCPPCP
Linker
(SEQ ID No. 7)
KDPK
IL18RAP transmembrane
(SEQ ID No. 12)
GVVLLYILLGTIGTLVAVLAA
IL18RAP endodomain
(SEQ ID No. 13)
SALLYRHWIEIVLLYRTYQSKDQTLGDKKDFDAFVSYAKWSSFPSEATS
SLSEEHLALSLFPDVLENKYGYSLCLLERDVAPGGVYAEDIVSIIKRSR
RGIFILSPNYVNGPSIFELQAAVNLALDDQTLKLILIKFCYFQEPESLP
HLVKKALRVLPTVTWRGLKSVPPNSRFWAKMRYHMPVKNSQGFTWNQLR
ITSRIFQWKGLSRTETTGRSSQPK

Another type of CCR which spontaneously dimerises (or multimerises) is one which comprises a coiled-coil domain.

A coiled coil is a structural motif in which two to seven alpha-helices are wrapped together like the strands of a rope. Coiled coils usually contain a repeated pattern, hxxhcxc, of hydrophobic (h) and charged (c) amino-acid residues, referred to as a heptad repeat. The packing in a coiled-coil interface is exceptionally tight, with almost complete van der Waals contact between the side-chains. Many endogenous proteins incorporate coiled coil domains, such as leucine zippers.

Leucine zippers are super-secondary structures that function as a dimerization domains. Their presence generates adhesion forces in parallel alpha helices. A single leucine zipper consists of multiple leucine residues at approximately 7-residue intervals, which forms an amphipathic alpha helix with a hydrophobic region running along one side. This hydrophobic region provides an area for dimerization, allowing the motifs to “zip” together. Leucine zippers are typically 20 to 40 amino acids in length, for example approximately 30 amino acids. Such leucine zippers are found in transcription factors, including c-fos and c-jun, which form the adaptor protein-1 transcription complex. The Jun-Fos leucine zipper has previously been used to dimerize scFv antibodies, interferon γ receptor β subunit and the receptors for the cytokines granulocyte-macrophage colony stimulating factor and growth hormone.

The first and/or second heterodimerization domain may comprise the sequence shown as SEQ ID NO: 54 or 55. The first heterodimerization domain may comprise the sequence shown as SEQ ID NO: 54 and the second heterodimerization domain may comprise the sequence shown as SEQ ID NO: 55, or vice versa.

SEQ ID NO: 54:
QLEKELQALEKENAQLEWELQALEKELAQ
SEQ ID NO: 55:
QLEKKLQALKKKNAQLKWKLQALKKKLAQ

In certain embodiments, the first and second heterodimerization domains may be acidic (e.g. SEQ ID NO: 54) or basic (e.g. SEQ ID NO: 55) leucine zippers. In particular, where the first heterodimerization domain is an acidic leucine zipper, the second heterodimerization is a basic leucine zipper and vice versa.

The first and second heterodimerization domains may be dimerization and docking domain (DDD1) and anchoring domain (AD1).

DDD1 is a short alpha helical structure derived from Protein Kinase A (PKA). AD1 is a short alpha helical structure derived from A-kinase anchor proteins (AKAPs).

The DDD1 domain may comprise the sequence shown as SEQ ID NO: 56.

SEQ ID NO. 56:
SHIQIPPGLTELLQGYTVEVLRQQPPDLVEFAVEYFTRLREARA

The AD1 domain may comprise the sequence shown as SEQ ID NO: 57

SEQ ID NO: 57:
VQIEYLAKQIVDNAIQQA.

The coiled coil domain may form a dimer or a multimer, in particular a multimer which is an order of two (e.g. 2, 4, 6 or 8 chains). The CCR may form a tetramer, for example by using the coiled coil domains from the SNAP-25/SNARE heterotetrametric complex. This complex comprises four chains: Chain A: Chain B; Chain C and Chain D, as shown below.

SNAP-25 and SNARE: parallel hetero-tetramer
Chain A:
(SEQ ID No. 58)
IETRHSEIIKLENSIRELHDMFMDMAMLVESQGEMIDRIEYNVEHAVDY
VE
Chain B:
(SEQ ID No. 59)
ALSEIETRHSEIIKLENSIRELHDMFMDMAMLVESQGEMIDRIEYNVEH
AVDYVERAVSDTKKAVKY
Chain C:
(SEQ ID No. 60)
ELEEMQRRADQLADESLESTRRMLQLVEESKDAGIRTLVMLDEQGEQLE
RIEEGMDQINKDMKEAEKNL
Chain D:
(SEQ ID NO. 61)
IETRHSEIIKLENSIRELHDMFMDMAMLVESQGEMIDRIEYNVEHAVDY
VE

By linking the tetramerization domains of two of these chains with IL18RAP endodomain and two of these chains with the IL18BR1 endodomain, as illustrated schematically in FIG. 11, two copies of IL18RAP endodomain are brought together with two copies of the IL18BR1 endodomain, giving constitutive IL-8 signalling.

An alternative constitutively active CCR is one in which the first or second polypeptide comprises an integral IL-18 molecule. Such an arrangement is illustrated schematically in FIG. 1A, in which the receptor comprises IL-18R α and β chains, and one of the chains also comprises an IL-18 molecule tethered to the polypeptide by way of a linker. The tethered IL-18 binds and dimerises the IL18R1 and IL18RAP chains, due to its permanent physical proximity, giving constitutive IL-18 signalling.

The sequence of human IL-18 is available from Uniprot accession No. Q14116 and shown below as SEQ ID No. 14. The CCR of the present invention may comprise a truncated version of IL-18 which lacks the signal sequence. Such a truncated sequence is shown below as SEQ ID No. 15.

(human IL-18)
SEQ ID No. 14
MAAEPVEDNCINFVAMKFIDNTLYFIAEDDENLESDYFGKLESKLSVIR
NLNDQVLFIDQGNRPLFEDMTDSDCRDNAPRTIFIISMYKDSQPRGMAV
TISVKCEKISTLSCENKIISFKEMNPPDNIKDTKSDIIFFQRSVPGHDN
KMQFESSSYEGYFLACEKERDLFKLILKKEDELGDRSIMFTVQNED
(truncated IL-18)
SEQ ID No. 15
YFGKLESKLSVIRNLNDQVLFIDQGNRPLFEDMTDSDCRDNAPRTIFII
SMYKDSQPRGMAVTISVKCEKISTLSCENKIISFKEMNPPDNIKDTKSD
IIFFQRSVPGHDNKMQFESSSYEGYFLACEKERDLFKLILKKEDELGDR
SIMFTVQNED

The IL-18 molecule, or truncated IL-18 molecule may be tethered to the first or second polypeptide. It may be N-terminal to the IL18R1 ectodomain or IL18RAP ectodomain. It may be tethered by way of a linker, such as a serine-glycine linker. The linker may, for example, be between 10 and 40 amino acids in length, for example between 20 and 30 or 24 to 28 amino acids in length.

Two illustrative sequences for creating a CCR as shown in FIG. 1A are shown below, with the individual components identified. In the first sequence the truncated IL-18 molecule is attached to the N-terminus of IL18R1 and the second sequence the truncated IL-18 molecule is attached to the N-terminus of IL18RAP.

### Tethered IL-18—Version 1

(SEQ ID NO. 9)
Signal peptide
(SEQ ID No. 11)
METDTLILWVLLLLVPGSTG
Truncated IL18
(SEQ ID No. 15)
YFGKLESKLSVIRNLNDQVLFIDQGNRPLFEDMTDSDCRDNAPRTIFII
SMYKDSQPRGMAVTISVKCEKISTLSCENKIISFKEMNPPDNIKDTKSD
IIFFQRSVPGHDNKMQFESSSYEGYFLACEKERDLFKLILKKEDELGDR
SIMFTVQNED
Linker
(SEQ ID No. 16)
SGGGGSGGGGSGGGGSGGGGSGGGGS
IL18R1 ectodomain
(SEQ ID No. 17)
CTSRPHITVVEGEPFYLKHCSCSLAHEIETTTKSWYKSSGSQEHVELNP
RSSSRIALHDCVLEFWPVELNDTGSYFFQMKNYTQKWKLNVIRRNKHSC
FTERQVTSKIVEVKKFFQITCENSYYQTLVNSTSLYKNCKKLLLENNKN
PTIKKNAEFEDQGYYSCVHFLHHNGKLFNITKTFNITIVEDRSNIVPVL
LGPKLNHVAVELGKNVRLNCSALLNEEDVIYWMFGEENGSDPNIHEEKE
MRIMTPEGKWHASKVLRIENIGESNLNVLYNCTVASTGGTD
TKSFILVRKADMADIPGHVFTR
IL18R1 transmembrane
(SEQ ID No. 8)
GMIIAVLILVAVVCLVTVCVI
IL18R1 endodomain
YRVDLVLFYRHLTRRDETLTDGKTYDAFVSYLKECRPENGEEHTFAVEI
LPRVLEKHFGYKLCIFERDVVPGGAVVDEIHSLIEKSRRLIIVLSKSYM
SNEVRYELESGLHEALVERKIKIILIEFTPVTDFTFLPQSLKLLKSHRV
LKWKADKSLSYNSRFWKNLLYLMPAKTVKPGRDEPEVLPVLSES

Signal peptide
(SEQ ID No. 11)
METDTLILWVLLLLVPGSTG
Truncated IL18
(SEQ ID No. 15)
YFGKLESKLSVIRNLNDQVLFIDQGNRPLFEDMTDSDCRDNAPRTIFII
SMYKDSQPRGMAVTISVKCEKISTLSCENKIISFKEMNPPDNIKDTKSD
IIFFQRSVPGHDNKMQFESSSYEGYFLACEKERDLFKLILKKEDELGDR
SIMFTVQNED
Linker
(SEQ ID No. 16)
SGGGGSGGGGGGGGSGGGGSGGGGS
IL18RAP ectodomain
(SEQ ID No. 18)
FNISGCSTKKLLWTYSTRSEEEFVLFCDLPEPQKSHFCHRNRLSPKQVP
EHLPFMGSNDLSDVQWYQQPSNGDPLEDIRKSYPHIIQDKCTLHFLTPG
VNNSGSYICRPKMIKSPYDVACCVKMILEVKPQTNASCEYSASHKQDLL
LGSTGSISCPSLSCQSDAQSPAVTWYKNGKLLSVERSNRIVVDEVYDYH
QGTYVCDYTQSDTVSSWTVRAVVQVRTIVGDTKLKPDILDPVEDTLEVE
LGKPLTISCKARFGFERVFNPVIKWYIKDSDLEWEVSVPEAKSIKSTLK
DEIIERNIILEKVTQRDLRRKFVCFVQNSIGNTTQSVQLKEKR
IL18RAP transmembrane
(SEQ ID No. 12)
GVVLLYILLGTIGTLVAVLAA
IL18RAP endodomain
(SEQ ID No. 13)
SALLYRHWIEIVLLYRTYQSKDQTLGDKKDFDAFVSYAKWSSFPSEATS
SLSEEHLALSLFPDVLENKYGYSLCLLERDVAPGGVYAEDIVSIIKRSR
RGIFILSPNYVNGPSIFELQAAVNLALDDQTLKLILIKFCYFQEPESLP
HLVKKALRVLPTVTWRGLKSVPPNSRFWAKMRYHMPVKNSQGFTWNQLR
ITSRIFQWKGLSRTETTGRSSQPK

In order to generate a CCR of the type illustrated in FIG. 1A, is only necessary to express the polypeptide chain recombinantly which contains the linked IL-18 molecule. This polypeptide will pair with the endogenous chain for the complementary part of the IL18 receptor to produce the CCR. So version 1 above, in which IL-18 is tethered via a linker to IL18R1, will pair with endogenous IL18RAP; and version 2 above in which IL-18 is tethered via a linker to IL18RAP, will pair with endogenous IL18R1.

### CID/Ligand-Activated Systems

As an alternative to constitutively active systems, dimerization may occur only in the presence of a chemical inducer of dimerization (CID) or ligand in which case the transmembrane protein only causes cytokine-type signalling in the presence of the CID or ligand.

Suitable dimerization domains and CIDs are described in WO2015/150771, the contents of which are hereby incorporated by reference.

For example, one dimerization domain may comprise the rapamycin binding domain of FK-binding protein 12 (FKBP12), the other may comprise the FKBP12-Rapamycin Binding (FRB) domain of mTOR; and the CID may be rapamycin or a derivative thereof.

One dimerization domain may comprise the FK506 (Tacrolimus) binding domain of FK-binding protein 12 (FKBP12) and the other dimerization domain may comprise the cyclosporin binding domain of cylcophilin A; and the CID may be an FK506/cyclosporin fusion or a derivative thereof.

One dimerization domain may comprise an oestrogen-binding domain (EBD) and the other dimerization domain may comprise a streptavidin binding domain; and the CID may be an estrone/biotin fusion protein or a derivative thereof.

One dimerization domain may comprise a glucocorticoid-binding domain (GBD) and the other dimerization domain may comprise a dihydrofolate reductase (DHFR) binding domain; and the CID may be a dexamethasone/methotrexate fusion protein or a derivative thereof.

One dimerization domain may comprise an 06-alkylguanine-DNA alkyltransferase (AGT) binding domain and the other dimerization domain may comprise a dihydrofolate reductase (DHFR) binding domain; and the CID may be an 06-benzylguanine derivative/methotrexate fusion protein or a derivative thereof.

One dimerization domain may comprise a retinoic acid receptor domain and the other dimerization domain may comprise an ecodysone receptor domain; and the CID may be RSL1 or a derivative thereof.

Rather than being activated by a chemical inducer of dimerization which is administered externally to the patient, the CCR may dimerise in the presence of a naturally occurring ligand, such as a tumour secreted factor or TGFβ.

In the arrangement illustrated schematically in FIG. 1B, the first and second polypeptides of the CCR each comprise a binding domain, such as an svFv or dAb, each of which bind a ligand. in the presence of the ligand, the exodomains both bind, bringing the endodomains together and giving ligand-dependent IL-18 signalling.

The ligand may, for example, be a tumour secreted factor or chemokine or cell surface antigen.

Numerous ligand-binding domains are known in the art, including those based on the antigen binding site of an antibody, antibody mimetics, and T-cell receptors. For example, the antigen-binding domain may comprise: a single-chain variable fragment (scFv) derived from a monoclonal antibody; the binding domain from a natural receptor for the target antigen; a peptide with sufficient affinity for the target ligand; a single domain binder such as a camelid; an artificial binder single as a Darpin; or a single-chain derived from a T-cell receptor.

The term “ligand” is used synonymously with “antigen” to mean an entity which is specifically recognised and bound by the antigen-binding domain of the CCR.

The ligand may be a tumour secreted factor such as prostate-specific antigen (PSA), carcinoembryonic antigen (CEA) and vascular endothelial growth factor (VEGF) or CA125.

Two illustrative sequences for creating a PSA-sensing CCR as shown in FIG. 1B are shown below, with the individual components identified. The PSA binding domains of the CCR are 5D3D11 and 5D5A5 which are described in WO2017/029512. In the first version, the 5D3D11 scFv is attached, via a spacer and transmembrane domain, to the IL18R1 endodomain; and the 5D5A5 scFv is attached, via a spacer and transmembrane domain, to the IL18RAP endodomain. In the second version, the 5D5A5 scFv is attached, via a spacer and transmembrane domain, to the IL18R1 endodomain; and the 5D3D11 scFv is attached, via a spacer and transmembrane domain, to the IL18RAP endodomain.

### PSA Sensing—Version 1

Signal peptide murine heavy chain
(SEQ ID No. 5)
MGWSCIILFLVATATGVHS
V5 tag
(SEQ ID No. 19)
DSSGKPIPNPLLGLDS
Linker
(SEQ ID No. 20)
SGGGGS
aPSA 5D3D11 light chain
(SEQ ID No. 21)
DIVMTQTAPSVFVTPGESVSISCRSSKSLLHSNGNTYLYWFLQRPGQSP
QLLIYRMSNLASGVPDRFSGSGSGTDFTLRISRVEAEDVGVYYCMQHLE
YPVTFGAGTKVEIKR
Linker
(SEQ ID No. 22)
SGGGGSGGGGSGGGGS
aPSA 5D3D11 heavy chain
(SEQ ID No. 23)
QVQLQQSGPELVKPGASVKISCKVSGYAISSSWMNWVKQRPGQGLEWIG
RIYPGDGDTKYNGKFKDKATLTVDKSSSTAYMQLSSLTSVDSAVYFCAR
DGYRYYFDYWGQGTSVTVSSDP
Monomeric CD8stk
(SEQ ID No. 24)
TTTPAPRPPTPAPTIASQPLSLRPEASRPAAGGAVHTRGLDFASDI
IL18R1 transmembrane
(SEQ ID No. 8)
GMIIAVLILVAVVCLVTVCVI
IL18R1 endodomain
(SEQ ID No. 9)
YRVDLVLFYRHLTRRDETLTDGKTYDAFVSYLKECRPENGEEHTFAVEI
LPRVLEKHFGYKLCIFERDVVPGGAVVDEIHSLIEKSRRLIIVLSKSYM
SNEVRYELESGLHEALVERKIKIILIEFTPVTDFTFLPQSLKLLKSHRV
LKWKADKSLSYNSRFWKNLLYLMPAKTVKPGRDEPEVLPVLSES
T2A
(SEQ ID No. 10)
EGRGSLLTCGDVEENPGP
Signal peptide murine heavy chain
(SEQ ID No. 5)
MGWSCIILFLVATATGVHS
HA tag
(SEQ ID No. 25)
YPYDVPDYA
Linker
(SEQ ID No. 20)
SGGGGS
aPSA 5D5A5 scFv heavy chain
(SEQ ID No. 26)
QVQLQQSGAELAKPGASVKMSCKTSGYSFSSYWMHWVKQRPGQGLEWIG
YINPSTGYTENNQKFKDKVTLTADKSSNTAYMQLNSLTSEDSAVYYCAR
SGRLYFDVWGAGTTVTVS
Linker
(SEQ ID No. 22)
SGGGGSGGGGSGGGGS
aPSA 5D5A5 scFv light chain
(SEQ ID No. 27)
DIVLTQSPPSLAVSLGQRATISCRASESIDLYGFTFMHWYQQKPGQPPK
ILIYRASNLESGIPARFSGSGSRTDFTLTINPVEADDVATYYCQQTHED
PYTFGGGTKLEIKRSDPA
Monomeric hinge spacer
(SEQ ID No. 28)
EPKSPDKTHTSPPSPKDPK
IL18RAP transmembrane
(SEQ ID No. 12)
GVVLLYILLGTIGTLVAVLAA
IL18RAP endodomain
(SEQ ID No. 13)
SALLYRHWIEIVLLYRTYQSKDQTLGDKKDFDAFVSYAKWSSFPSEATS
SLSEEHLALSLFPDVLENKYGYSLCLLERDVAPGGVYAEDIVSIIKRSR
RGIFILSPNYVNGPSIFELQAAVNLALDDQTLKLILIKFCYFQEPESLP
HLVKKALRVLPTVTWRGLKSVPPNSRFWAKMRYHMPVKNSQGFTWNQLR
ITSRIFQWKGLSRTETTGRSSQPK

### PSA Sensing—Version 2

Signal peptide murine heavy chain
(SEQ ID No. 5)
MGWSCIILFLVATATGVHS
V5 tag
(SEQ ID No. 19)
DSSGKPIPNPLLGLDS
Linker
(SEQ ID No. 20)
SGGGGS
aPSA 5D3D11 light chain
(SEQ ID No. 21)
DIVMTQTAPSVFVTPGESVSISCRSSKSLLHSNGNTYLYWFLQRPGQSP
QLLIYRMSNLASGVPDRFSGSGSGTDFTLRISRVEAEDVGVYYCMQHLE
YPVTFGAGTKVEIKR
Linker
(SEQ ID No. 22)
SGGGGSGGGGSGGGGS
aPSA 5D3D11 heavy chain
(SEQ ID No. 23)
QVQLQQSGPELVKPGASVKISCKVSGYAISSSWMNWVKQRPGQGLEWIG
RIYPGDGDTKYNGKFKDKATLTVDKSSSTAYMQLSSLTSVDSAVYFCAR
DGYRYYFDYWGQGTSVTVSSDP
Monomeric CD8stk
(SEQ ID No. 24)
TTTPAPRPPTPAPTIASQPLSLRPEASRPAAGGAVHTRGLDFASDI
IL18RAP transmembrane
(SEQ ID No. 12)
GVVLLYILLGTIGTLVAVLAA
IL18RAP endodomain
(SEQ ID No. 13)
SALLYRHWIEIVLLYRTYQSKDQTLGDKKDFDAFVSYAKWSSFPSEATS
SLSEEHLALSLFPDVLENKYGYSLCLLERDVAPGGVYAEDIVSIIKRSR
RGIFILSPNYVNGPSIFELQAAVNLALDDQTLKLILIKFCYFQEPESLP
HLVKKALRVLPTVTWRGLKSVPPNSRFWAKMRYHMPVKNSQGFTWNQLR
ITSRIFQWKGLSRTETTGRSSQPK
T2A
(SEQ ID No. 10)
EGRGSLLTCGDVEENPGP
Signal peptide murine heavy chain
(SEQ ID No. 5)
MGWSCIILFLVATATGVHS
HA tag
(SEQ ID No. 25)
YPYDVPDYA
Linker
(SEQ ID No. 20)
SGGGGS
aPSA 5D5A5 scFv heavy chain
(SEQ ID No. 26)
QVQLQQSGAELAKPGASVKMSCKTSGYSFSSYWMHWVKQRPGQGLEWIG
YINPSTGYTENNQKFKDKVTLTADKSSNTAYMQLNSLTSEDSAVYYCAR
SGRLYFDVWGAGTTVTVS
Linker
(SEQ ID No. 22)
SGGGGSGGGGSGGGGS
aPSA 5D5A5 scFv light chain
(SEQ ID No. 27)
DIVLTQSPPSLAVSLGQRATISCRASESIDLYGFTFMHWYQQKPGQPPK
ILIYRASNLESGIPARFSGSGSRTDFTLTINPVEADDVATYYCQQTHED
PYTFGGGTKLEIKRSDPA
Monomeric hinge spacer
(SEQ ID No. 28)
EPKSPDKTHTSPPSPKDPK
IL18R1 transmembrane
(SEQ ID No. 8)
GMIIAVLILVAVVCLVTVCVI
IL18R1 endodomain
(SEQ ID No. 9)
YRVDLVLFYRHLTRRDETLTDGKTYDAFVSYLKECRPENGEEHTFAVEI
LPRVLEKHFGYKLCIFERDVVPGGAVVDEIHSLIEKSRRLIIVLSKSYM
SNEVRYELESGLHEALVERKIKIILIEFTPVTDFTFLPQSLKLLKSHRV
LKWKADKSLSYNSRFWKNLLYLMPAKTVKPGRDEPEVLPVLSES

The CCR of the present invention may be triggered by the presence of TGFβ in the extracellular milieu. For example, in the arrangement illustrated schematically in FIG. 1C, the first and second polypeptides of the CCR each comprise an exodomain from a TGFβ receptor. For example, one polypeptide may comprise an exodomain from TGFβ receptor I and the other polypeptide may comprise an exodomain from TGFβ receptor II. In the presence of TGFβ, the two TGFβ receptor endodomains come together causing IL-18-type signalling.

Two illustrative sequences for creating a TGFβ-triggered CCR as shown in FIG. 1C are shown below, with the individual components identified. In the first version, the TGFβR2 exodomain is attached, via a spacer and transmembrane domain, to the IL18R1 endodomain; and the TGFβR1 exodomain is attached, via a spacer and transmembrane domain, to the IL18RAP endodomain. In the second version, the TGFβR1 exodomain is attached, via a spacer and transmembrane domain, to the IL18RAP endodomain; and the TGFβR2 exodomain is attached, via a spacer and transmembrane domain, to the IL18R1 endodomain.

### TGFbeta-IL18 Switch Receptor: Version 1

Signal peptide
(SEQ ID No. 5)
MGWSCIILFLVATATGVHS
TGFbetaR2 ectodomain
(SEQ ID No. 29)
TIPPHVQKSVNNDMIVTDNNGAVKFPQLCKFCDVRFSTCDNQKSCMSNC
SITSICEKPQEVCVAVWRKNDENITLETVCHDPKLPYHDFILEDAASPK
CIMKEKKKPGETFFMCSCSSDECNDNIIFSEEYNTSNPDLLLVIFQ
TGFbetaR2 transmembrane
(SEQ ID No. 30)
ISLLPPLGVAISVIIIFY
IL18R1 endodomain
(SEQ ID No. 9)
YRVDLVLFYRHLTRRDETLTDGKTYDAFVSYLKECRPENGEEHTFAVEI
LPRVLEKHFGYKLCIFERDVVPGGAVVDEIHSLIEKSRRLIIVLSKSYM
SNEVRYELESGLHEALVERKIKIILIEFTPVTDFTFLPQSLKLLKSHRV
LKWKADKSLSYNSRFWKNLLYLMPAKTVKPGRDEPEVLPVLSES
T2A
(SEQ ID No. 10)
EGRGSLLTCGDVEENPGP
Murine heavy chain signal peptide
(SEQ ID No. 5)
MGWSCIILFLVATATGVHS
TGFbetaR1 ectodomain
(SEQ ID No. 31)
LQCFCHLCTKDNFTCVTDGLCFVSVTETTDKVIHNSMCIAEIDLIPRDR
PFVCAPSSKTGSVTTTYCCNQDHCNKIELPTTVKSSPGLGPVE
TGFbetaR1 transmembrane
(SEQ ID NO. 32)
LAAVIAGPVCFVCISLMLMVYI
IL18RAP endodomain
(SEQ ID No. 13)
SALLYRHWIEIVLLYRTYQSKDQTLGDKKDFDAFVSYAKWSSFPSEATS
SLSEEHLALSLFPDVLENKYGYSLCLLERDVAPGGVYAEDIVSIIKRSR
RGIFILSPNYVNGPSIFELQAAVNLALDDQTLKLILIKFCYFQEPESLP
HLVKKALRVLPTVTWRGLKSVPPNSRFWAKMRYHMPVKNSQGFTWNQLR
ITSRIFQWKGLSRTETTGRSSQPK

### TGFbeta-IL18 Switch Receptor—Version 2

Signal peptide
(SEQ ID No. 5)
MGWSCIILFLVATATGVHS
TGFbetaR2 ectodomain
(SEQ ID No. 29)
TIPPHVQKSVNNDMIVTDNNGAVKFPQLCKFCDVRFSTCDNQKSCMSNC
SITSICEKPQEVCVAVWRKNDENITLETVCHDPKLPYHDFILEDAASPK
CIMKEKKKPGETFFMCSCSSDECNDNIIFSEEYNTSNPDLLLVIFQ
TGFbetaR2 transmembrane
(SEQ ID No. 30)
VTGISLLPPLGVAISVIIIFY
IL18RAP endodomain
(SEQ ID No. 13)
SALLYRHWIEIVLLYRTYQSKDQTLGDKKDFDAFVSYAKWSSFPSEATS
SLSEEHLALSLFPDVLENKYGYSLCLLERDVAPGGVYAEDIVSIIKRSR
RGIFILSPNYVNGPSIFELQAAVNLALDDQTLKLILIKFCYFQEPESLP
HLVKKALRVLPTVTWRGLKSVPPNSRFWAKMRYHMPVKNSQGFTWNQLR
ITSRIFQWKGLSRTETTGRSSQPK
T2A
(SEQ ID No. 10)
EGRGSLLTCGDVEENPGP
Murine heavy chain signal peptide
(SEQ ID No. 5)
MGWSCIILFLVATATGVHS
TGFbetaR1 ectodomain
(SEQ ID No. 31)
LQCFCHLCTKDNFTCVTDGLCFVSVTETTDKVIHNSMCIAEIDLIPRDR
PFVCAPSSKTGSVTTTYCCNQDHCNKIELPTTVKSSPGLGPVE
TGFbetaR1 transmembrane
(SEQ ID No. 32)
LAAVIAGPVCFVCISLMLMVYI
IL18R1 endodomain
(SEQ ID NO. 9)
YRVDLVLFYRHLTRRDETLTDGKTYDAFVSYLKECRPENGEEHTFAVEI
LPRVLEKHFGYKLCIFERDVVPGGAVVDEIHSLIEKSRRLIIVLSKSYM
SNEVRYELESGLHEALVERKIKIILIEFTPVTDFTFLPQSLKLLKSHRV
LKWKADKSLSYNSRFWKNLLYLMPAKTVKPGRDEPEVLPVLSES

The arrangement illustrated schematically in FIG. 1D is also triggered by the presence of TGFβ. In this arrangement, one of the polypeptides comprises an ITAM-containing endodomain and an antigen-binding domain. This polypeptide acts like a CAR in that it induces activation of the cell in the presence of the target antigen. This polypeptide also comprises one of the IL-18 receptor endodomains and a TGFβ receptor exodomain. The other polypeptide in the CCR comprises a second TGFβ receptor exodomain and the other IL-18 receptor endodomain chain. In the presence of TGFβ, the two TGFβ receptor endodomains come together causing IL-18-type signalling. In the presence of both TGFβ and target antigen, the cell receives both an activation signal and an IL-18-type proliferation signal.

Two illustrative sequences for creating such a TGFbeta-IL18 CAR enhancer are shown below, with the individual components identified. The system includes an anti-EGFRvII domain antibody (dAb) as the antigen binding domain, so EGFRvII can be used as a model target antigen. Equivalent systems can be created targeting other target antigens by swapping in dAbs or scFvs with other binding specificities. In the first version sequence shown below, the anti-EGFRvIII dAb and TGFβR2 exodomain are attached, via a spacer and transmembrane domain, to the IL18R1 and CD3z endodomains; and the TGFβR1 exodomain is attached, via a spacer and transmembrane domain, to the IL18RAP endodomain. In the second version, the anti-EGFRvIII dAb and TGFβR1 exodomain are attached, via a spacer and transmembrane domain, to the IL18RAP and CD3z endodomains; and the TGFβR2 exodomain is attached, via a spacer and transmembrane domain, to the IL18R1 endodomain.

### TGFbeta-IL18 CAR Enhancer—Version 1

Signal peptide
(SEQ ID No. 5)
MGWSCIILFLVATATGVHS
aEGFRvIII Dab
(SEQ ID No. 33)
EVQLVESGGGLVQAGGSLRLSCAASGRTFSSYAMGWFRQAPGKEREFVV
AINWSSGSTYYADSVKGRFTISRDNAKNTMYLQMNSLKPEDTAVYYCAA
GYQINSGNYNFKDYEYDYWGQGTQVTVSSR
Linker
(SEQ ID No. 22)
SGGGGSGGGGSGGGGS
TGFbetaR2 ectodomain
(SEQ ID No. 29)
TIPPHVQKSVNNDMIVTDNNGAVKFPQLCKFCDVRFSTCDNQKSCMSNC
SITSICEKPQEVCVAVWRKNDENITLETVCHDPKLPYHDFILEDAASPK
CIMKEKKKPGETFFMCSCSSDECNDNIIFSEEYNTSNPDLLLVIFQ
TGFbetaR2 transmembrane
(SEQ ID No. 30)
VTGISLLPPLGVAISVIIIFY
IL18R1 endodomain
(SEQ ID No. 9)
YRVDLVLFYRHLTRRDETLTDGKTYDAFVSYLKECRPENGEEHTFAVEI
LPRVLEKHFGYKLCIFERDVVPGGAVVDEIHSLIEKSRRLIIVLSKSYM
SNEVRYELESGLHEALVERKIKIILIEFTPVTDFTFLPQSLKLLKSHRV
LKWKADKSLSYNSRFWKNLLYLMPAKTVKPGRDEPEVLPVLSES
TCRzeta
(SEQ ID No. 34)
RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPRRA
T2A
(SEQ ID No. 10)
EGRGSLLTCGDVEENPGP
Murine heavy chain signal peptide
(SEQ ID No. 5)
MGWSCIILFLVATATGVHS
TGFbetaR1 ectodomain
(SEQ ID No. 31)
LQCFCHLCTKDNFTCVTDGLCFVSVTETTDKVIHNSMCIAEIDLIPRDR
PFVCAPSSKTGSVTTTYCCNQDHCNKIELPTTVKSSPGLGPVE
TGFbetaR1 transmembrane
(SEQ ID No. 32)
LAAVIAGPVCFVCISLMLMVYI
IL18RAP endodomain
(SEQ ID No. 13)
SALLYRHWIEIVLLYRTYQSKDQTLGDKKDFDAFVSYAKWSSFPSEATS
SLSEEHLALSLFPDVLENKYGYSLCLLERDVAPGGVYAEDIVSIIKRSR
RGIFILSPNYVNGPSIFELQAAVNLALDDQTLKLILIKFCYFQEPESLP
HLVKKALRVLPTVTWRGLKSVPPNSRFWAKMRYHMPVKNSQGFTWNQLR
ITSRIFQWKGLSRTETTGRSSQPK

### TGFbeta-IL18 CAR Enhancer—Version 2

Signal peptide
(SEQ ID No. 5)
MGWSCIILFLVATATGVHS
aEGFRvIII Dab
(SEQ ID No. 33)
EVQLVESGGGLVQAGGSLRLSCAASGRTFSSYAMGWFRQAPGKEREFVV
AINWSSGSTYYADSVKGRFTISRDNAKNTMYLQMNSLKPEDTAVYYCAA
GYQINSGNYNFKDYEYDYWGQGTQVTVSSR
Linker
(SEQ ID No. 22)
SGGGGSGGGGSGGGGS
TGFbetaR2 ectodomain
(SEQ ID No. 29)
TIPPHVQKSVNNDMIVTDNNGAVKFPQLCKFCDVRFSTCDNQKSCMSNC
SITSICEKPQEVCVAVWRKNDENITLETVCHDPKLPYHDFILEDAASPK
CIMKEKKKPGETFFMCSCSSDECNDNIIFSEEYNTSNPDLLLVIFQ
TGFbetaR2 transmembrane
(SEQ ID No. 30)
VTGISLLPPLGVAISVIIIFY
IL18RAP endodomain
(SEQ ID No. 13)
SALLYRHWIEIVLLYRTYQSKDQTLGDKKDFDAFVSYAKWSSFPSEATS
SLSEEHLALSLFPDVLENKYGYSLCLLERDVAPGGVYAEDIVSIIKRSR
RGIFILSPNYVNGPSIFELQAAVNLALDDQTLKLILIKFCYFQEPESLP
HLVKKALRVLPTVTWRGLKSVPPNSRFWAKMRYHMPVKNSQGFTWNQLR
ITSRIFQWKGLSRTETTGRSSQPK
TCRzeta
(SEQ ID No. 34)
RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPRRA
T2A
(SEQ ID No. 10)
EGRGSLLTCGDVEENPGP
Murine heavy chain signal peptide
(SEQ ID No. 5)
MGWSCIILFLVATATGVHS
TGFbetaR1 ectodomain
(SEQ ID No. 31)
LQCFCHLCTKDNFTCVTDGLCFVSVTETTDKVIHNSMCIAEIDLIPRDR
PFVCAPSSKTGSVTTTYCCNQDHCNKIELPTTVKSSPGLGPVE
TGFbetaR1 transmembrane
(SEQ ID No. 32)
LAAVIAGPVCFVCISLMLMVYI
IL18R1 endodomain
(SEQ ID No. 9)
YRVDLVLFYRHLTRRDETLTDGKTYDAFVSYLKECRPENGEEHTFAVEI
LPRVLEKHFGYKLCIFERDVVPGGAVVDEIHSLIEKSRRLIIVLSKSYM
SNEVRYELESGLHEALVERKIKIILIEFTPVTDFTFLPQSLKLLKSHRV
LKWKADKSLSYNSRFWKNLLYLMPAKTVKPGRDEPEVLPVLSES
SPACER

The chimeric cytokine receptor of the present invention, in particular the ligand-activated version illustrated schematically in FIG. 1B, may comprise a spacer to connect the ligand-binding domains with the transmembrane domain and spatially separate the ligand-binding domain from the endodomain. A flexible spacer allows to the ligand-binding domain to orient in different directions to enable antigen binding.

Where the cell of the present invention comprises two or more chimeric cytokine receptors, the spacers may be the same or different. Where the cell of the present invention comprises a chimeric cytokine receptor (CCR) and a chimeric antigen receptor (CAR), the spacer of the CCR and the CAR may be different, for example, having a different length. The spacer of the CAR may be longer than the spacer of the or each CCR.

The spacer sequence may, for example, comprise an IgG1 Fc region, an IgG1 hinge or a CD8 stalk. The linker may alternatively comprise an alternative linker sequence which has similar length and/or domain spacing properties as an IgG1 Fc region, an IgG1 hinge or a CD8 stalk.

A human IgG1 spacer may be altered to remove Fc binding motifs.

The spacer may be modified to remove cysteine residues, so that it is monomeric. A sequence for monomeric CD8 stalk is shown above as SEQ ID No. 24

### Transmembrane Domain

The transmembrane domain is the sequence of a CCR that spans the membrane. It may comprise a hydrophobic alpha helix. The transmembrane domain may be derived from CD28, which gives good receptor stability.

The constitutively active CCR illustrated schematically in FIGS. 1A and 1E and the ligand-activated CCR illustrated schematically in FIG. 1B may comprise the IL18R1 and IL18RAP transmembrane domains which are shown above as SEQ ID No. 8 and 12 respectively. Alternatively, the TM domains of one or both chains may be swapped for a different TM domain sequence.

The TGFβ-activated CCR systems illustrated schematically in FIGS. 1C and D may comprise the TGFβ 1 and TGFβ 2 transmembrane domains which are shown above as SEQ ID No. 32 and 30 respectively. Alternatively, the TM domains of one or both chains may be swapped for a different TM domain sequence.

### Ligand

The first and second polypeptides of the CCR-type illustrated schematically in FIG. 1B specifically bind a ligand.

The ligand may be a soluble ligand such as a tumour secreted factor or a chemokine.

Alternatively, the ligand may be a membrane bound ligand, such as a cell surface antigen.

The term “soluble ligand” is used to indicate a ligand or antigen which is not part of or attached to a cell but which moves freely in the extracellular space, for example in a bodily fluid of the tissue of interest. The soluble ligand may exist in a cell-free state in the serum, plasma or other bodily fluid of an individual.

The soluble ligand may be associated with the presence or pathology of a particular disease, such as cancer.

The soluble ligand may be part of the cancer secretome, i.e. the collection of factors secreted by a tumour, be it from cancer stem cells, non-stem cells or the surrounding stroma. The soluble ligand may be secreted or shed by tumour cells (see next section).

The soluble ligand may be characteristic of a disease or of diseased tissue. It may be found exclusively, or at a higher level in a subject having the disease vs a healthy subject; or in diseased tissue vs healthy tissue. The soluble ligand may be expressed at at least a 2-fold, 5-fold, 10-fold, 100-fold, 1000-fold, 10,000-fold or 100,000 fold higher level a subject having the disease vs a healthy subject; or in diseased tissue vs healthy tissue.

The terms “cell-surface antigen” and “cell-surface ligand” is used synonymously with “membrane-bound antigen” and “membrane-bound ligand” to mean a ligand which is attached to or expressed on the surface of the cell. The cell-surface ligand may, for example, be a transmembrane protein.

The cell on which the cell-surface ligand is found may be a target cell, such as a cancer cell.

The cell-surface ligand may be associated with the presence or pathology of a particular disease, such as cancer. Alternatively, the cell-surface ligand may be characteristic of the cell type of the target cell (e.g. B-cell) without being necessarily associated with the diseased state.

Where the cell-surface ligand is characteristic of a disease or of diseased tissue it may be found exclusively, or at a higher level on the relevant cells a subject having the disease vs a healthy subject; or in diseased tissue vs healthy tissue. The cell-surface ligand may be expressed at at least a 2-fold, 5-fold, 10-fold, 100-fold, 1000-fold, 10,000-fold or 100,000 fold higher level on a cell of a subject having the disease vs a healthy subject; or in diseased tissue vs healthy tissue.

### Tumour Secreted Factor

The ligand recognised by the CCR may be a soluble ligand secreted by or shedded from a tumour.

This “tumour secreted factor” may, for example, be prostate-specific antigen (PSA), carcinoembryonic antigen (CEA), vascular endothelial growth factor (VEGF) or Cancer Antigen-125 (CA-125).

The tumour secreted factor may be a soluble ligand which is not a cytokine. The CCR of the present invention therefore grafts the binding specificity for a non-cytokine ligand on to the endodomain of a cytokine receptor.

### Prostate-Specific Antigen (PSA)

The soluble ligand may be prostate-specific antigen (PSA).

Prostate-specific antigen (PSA), also known as gamma-seminoprotein or kallikrein-3 (KLK3), is a glycoprotein enzyme encoded in humans by the KLK3 gene. PSA is a member of the kallikrein-related peptidase family and is secreted by the epithelial cells of the prostate gland.

PSA is present in small quantities in the serum of men with healthy prostates, but is elevated in individuals with prostate cancer and other prostate disorders.

PSA is a 237-residue glycoprotein and is activated by KLK2. Its physiological role is the liquefaction of the coagulum components of the semen leading to liberation of spermatozoa. In cancer, PSA may participate in the processes of neoplastic growth and metastasis.

PSA is a chymotrypsin-like serine protease with a typical His-Asp-Ser triad and a catalytic domain similar to those of other kallikrein-related peptidases. The crystal structure of PSA has been obtained i) in complex with the monoclonal antibody (mAb) 8G8F5 and ii) in a sandwich complex with two mAbs 5D5A5 and 5D3D11 (Stura et al (J. Mol. Biol. (2011) 414:530-544).

Various monoclonal antibodies are known, including clones 2G2-B2, 2D8-E8, IgG1/K described in Bavat et al Avicenna J. Med. Biotechnol. 2015, 7:2-7; and Leinonen (2004) 289:157-67.

The CCR of the present invention may, for example, comprise the 6 CDRs or the VH and/or VL domain(s) from a PSA-binding mAb such as 8G8F5, 5D5A5 or 5D3D11.

Where the CCR comprises two antigen binding specificities, binding different epitopes on PSA, one may be based on, for example 5D3D11 and one may be based on, for example, 5D5A5.

The amino acid sequences for 5D3D11 and 5D5A5 VH and VL are shown above as SEQ ID Nos. 21, 23, 26 and 27.

### Carcinoembryonic Antigen (CEA)

The soluble ligand may be CEA.

Carcinoembryonic antigen (CEA) describes a set of highly related glycoproteins involved in cell adhesion. CEA is normally produced in gastrointestinal tissue during fetal development, but the production stops before birth. Therefore CEA is usually present only at very low levels in the blood of healthy adults. However, the serum levels are raised in some types of cancer, which means that it can be used as a tumor marker in clinical tests.

CEA are glycosyl phosphatidyl inositol (GPI) cell surface anchored glycoproteins whose specialized sialofucosylated glycoforms serve as functional colon carcinoma L-selectin and E-selectin ligands, which may be critical to the metastatic dissemination of colon carcinoma cells. Immunologically they are characterized as members of the CD66 cluster of differentiation.

CEA and related genes make up the CEA family belonging to the immunoglobulin superfamily. In humans, the carcinoembryonic antigen family consists of 29 genes, 18 of which are normally expressed. The following is a list of human genes which encode carcinoembryonic antigen-related cell adhesion proteins: CEACAM1, CEACAM3, CEACAM4, CEACAM5, CEACAM6, CEACAM7, CEACAM8, CEACAM16, CEACAM18, CEACAM19, CEACAM20, CEACAM21

Various antibodies which target CEA are described in WO 2011/034660.

A cell expressing a CCR against CEA may be useful in the treatment of, for example, colorectal cancer.

### Vascular Endothelial Growth Factor (VEGF)

The soluble ligand may be VEGF.

Vascular endothelial growth factor (VEGF) is a signal protein produced by cells that stimulates vasculogenesis and angiogenesis. It is part of the system that restores the oxygen supply to tissues when blood circulation is inadequate. Serum concentration of VEGF is high in bronchial asthma and diabetes mellitus. VEGF's normal function is to create new blood vessels during embryonic development, new blood vessels after injury, muscle following exercise, and new vessels (collateral circulation) to bypass blocked vessels.

When VEGF is overexpressed, it can contribute to disease. Solid cancers cannot grow beyond a limited size without an adequate blood supply; cancers that can express VEGF are able to grow and metastasize.

VEGF is a sub-family of the platelet-derived growth factor family of cystine-knot growth factors. They are important signaling proteins involved in both vasculogenesis (the de novo formation of the embryonic circulatory system) and angiogenesis (the growth of blood vessels from pre-existing vasculature).

The VEGF family comprises in mammals five members: VEGF-A, placenta growth factor (PGF), VEGF-B, VEGF-C and VEGF-D.

Various antibodies to VEGF are known, such as bevacizumab (Avastin) and Ranibizumab (Lucentis).

### Cancer Antigen 125 (CA-125)

CA-125 is associated with ovarian cancer and is the most frequently used biomarker for ovarian cancer detection. While CA-125 is best known as a marker for ovarian cancer, it may also be elevated in other cancers, including endometrial cancer, fallopian tube cancer, lung cancer, breast cancer and gastrointestinal cancer.

The sequence of human CA-125 (also known as mucin-16) is available from NCBI, Accession No. 078966.

A number of CA125-binding monoclonal antibodies are known, including OC125 and M11 (Nustad et al 1996, Tumour Biol. 17:196-329). In this study the specificity of 26 monoclonal antibodies against the CA 125 antigen was investigated. It was found that the CA 125 antigen carries only two major antigenic domains, which classifies the antibodies as OC125-like (group A) or M11-like (group B).

The chimeric cytokine receptor of the present invention may comprise an antigen-binding domain from such an antibody. A cell comprising such a CCR may be useful in the treatment of, for example, ovarian cancer.

The tumour secreted factor (or, if in a membrane-bound form, the transmembrane protein) may be selected from the following non-exhaustive list:
ALK gene rearrangements and overexpression giving mutated forms of ALK proteins
Alpha-fetoprotein (AFP)
Beta-2-microglobulin (B2M)
Beta-human chorionic gonadotropin (Beta-hCG)
BRAF V600 mutations giving mutated B-REF protein
C-kit/CD117
CA15-3/CA27.29
CA19-9
Calcitonin
CD20
Chromogranin A (CgA)
Cytokeratin fragment 21-1
EGFR gene mutation analysis
Estrogen receptor (ER)/progesterone receptor (PR)
Fibrin/fibrinogen
HE4
HER2/neu gene amplification or protein overexpression
Immunoglobulins
KRAS gene mutation analysis
Lactate dehydrogenase
Neuron-specific enolase (NSE)
Nuclear matrix protein 22
Programmed death ligand 1 (PD-L1)
Thyroglobulin
Urokinase plasminogen activator (uPA) and plasminogen activator inhibitor (PAI-1)

### Chemokine

Chemokines are chemotactic cytokines. Cell migration is guided by chemokine gradients embedded and immobilized in extracellular matrix. The positively charged chemokines like CXCL12 bind to negatively charged ECM molecules. These gradients provide tracks for cancer cell and immune cell homing. The action on T cells seems to be inhibitory for the homing of cytotoxic T cells, while regulatory T cells appear to be attracted.

Chemokines are approximately 8-10 kilodaltons in mass and have four cysteine residues in conserved locations which are key to forming their 3-dimensional shape.

Some chemokines are considered pro-inflammatory and can be induced during an immune response to recruit cells of the immune system to a site of infection, while others are considered homeostatic and are involved in controlling the migration of cells during normal processes of tissue maintenance or development.

Chemokines have been classified into four main subfamilies: CXC, CC, CX3C and XC. All of these proteins exert their biological effects by interacting with G protein-linked transmembrane receptors called chemokine receptors that are selectively found on the surfaces of their target cells.

The major role of chemokines is to act as a chemoattractant to guide the migration of cells. Cells that are attracted by chemokines follow a signal of increasing chemokine concentration towards the source of the chemokine. Some chemokines control cells of the immune system during processes of immune surveillance, such as directing lymphocytes to the lymph nodes so they can screen for invasion of pathogens by interacting with antigen-presenting cells residing in these tissues. Other chemokines are inflammatory and are released from a wide variety of cells in response to bacterial infection, viruses and other agents. Their release is often stimulated by pro-inflammatory cytokines such as interleukin 1. Inflammatory chemokines function mainly as chemoattractants for leukocytes, recruiting monocytes, neutrophils and other effector cells from the blood to sites of infection or tissue damage. Certain inflammatory chemokines activate cells to initiate an immune response or promote wound healing. They are released by many different cell types and serve to guide cells of both innate immune system and adaptive immune system.

### CC Chemokines

The CC chemokine (or β-chemokine) proteins have two adjacent cysteines (amino acids), near their amino terminus. There have been at least 27 distinct members of this subgroup reported for mammals, called CC chemokine ligands (CCL)-1 to -28; CCL10 is the same as CCL9. Chemokines of this subfamily usually contain four cysteines (C4-CC chemokines), but a small number of CC chemokines possess six cysteines (C6-CC chemokines). C6-CC chemokines include CCL1, CCL15, CCL21, CCL23 and CCL28. CC chemokines induce the migration of monocytes and other cell types such as NK cells and dendritic cells.

Examples of CC chemokine include monocyte chemoattractant protein-1 (MCP-1 or CCL2) which induces monocytes to leave the bloodstream and enter the surrounding tissue to become tissue macrophages.

CCL5 (or RANTES) attracts cells such as T cells, eosinophils and basophils that express the receptor CCR5.

### CXC Chemokines

The two N-terminal cysteines of CXC chemokines (or α-chemokines) are separated by one amino acid, represented in this name with an “X”. There have been 17 different CXC chemokines described in mammals, that are subdivided into two categories, those with a specific amino acid sequence (or motif) of glutamic acid-leucine-arginine (or ELR for short) immediately before the first cysteine of the CXC motif (ELR-positive), and those without an ELR motif (ELR-negative). ELR-positive CXC chemokines specifically induce the migration of neutrophils, and interact with chemokine receptors CXCR1 and CXCR2.

### C Chemokines

The third group of chemokines is known as the C chemokines (or γ chemokines), and is unlike all other chemokines in that it has only two cysteines; one N-terminal cysteine and one cysteine downstream. Two chemokines have been described for this subgroup and are called XCL1 (lymphotactin-α) and XCL2 (lymphotactin-β).

### CX3C Chemokine

CX3C chemokines have three amino acids between the two cysteines. The only CX3C chemokine discovered to date is called fractalkine (or CX3CL1). It is both secreted and tethered to the surface of the cell that expresses it, thereby serving as both a chemoattractant and as an adhesion molecule.

Chemokine receptors are G protein-coupled receptors containing 7 transmembrane domains that are found on the surface of leukocytes. Approximately 19 different chemokine receptors have been characterized to date, which are divided into four families depending on the type of chemokine they bind; CXCR that bind CXC chemokines, CCR that bind CC chemokines, CX3CR1 that binds the sole CX3C chemokine (CX3CL1), and XCR1 that binds the two XC chemokines (XCL1 and XCL2). They share many structural features; they are similar in size (with about 350 amino acids), have a short, acidic N-terminal end, seven helical transmembrane domains with three intracellular and three extracellular hydrophilic loops, and an intracellular C-terminus containing serine and threonine residues important for receptor regulation. The first two extracellular loops of chemokine receptors each has a conserved cysteine residue that allow formation of a disulfide bridge between these loops. G proteins are coupled to the C-terminal end of the chemokine receptor to allow intracellular signaling after receptor activation, while the N-terminal domain of the chemokine receptor determines ligand binding specificity.

### CXCL12

CXCL12 is strongly chemotactic for lymphocytes. CXCL12 plays an important role in angiogenesis by recruiting endothelial progenitor cells (EPCs) from the bone marrow through a CXCR4 dependent mechanism. It is this function of CXCL12 that makes it a very important factor in carcinogenesis and the neovascularisation linked to tumour progression. CXCL12 also has a role in tumour metastasis where cancer cells that express the receptor CXCR4 are attracted to metastasis target tissues that release the ligand, CXCL12.

The receptor for CXCL12 is CXCR4. The CCR of the present invention may comprise the CXCL12-binding domain from CXCR4 linked to an endodomain derived from a cytokine receptor, such as the IL-2 receptor.

CXCR4 coupled expression of IL2 would support engraftment of therapeutic T cell for cancer therapies. In multiple myeloma, a cell expressing such a CCR may mobilize cells and change the bone marrow environment. Such cells also have uses in the treatment of solid cancers by modifying the solid tumour microenvironment.

CXCR7 also binds CXCL12.

### CCL2

The chemokine (C-C motif) ligand 2 (CCL2) is also referred to as monocyte chemotactic protein 1 (MCP1) and small inducible cytokine A2. CCL2 recruits monocytes, memory T cells, and dendritic cells to the sites of inflammation produced by either tissue injury or infection.

CCR2 and CCR4 are two cell surface receptors that bind CCL2.

The CCR of the present invention may comprise the CCL2 binding site of CCR2 or CCR4 in its ligand binding domain.

### Cell-Surface Antigen

The ligand may be a cell-surface antigen, such as a transmembrane protein.

The cell surface antigen may be CD22.

CD22, or cluster of differentiation-22, is a molecule belonging to the SIGLEC family of lectins. It is found on the surface of mature B cells and to a lesser extent on some immature B cells. Generally speaking, CD22 is a regulatory molecule that prevents the overactivation of the immune system and the development of autoimmune diseases.

CD22 is a sugar binding transmembrane protein, which specifically binds sialic acid with an immunoglobulin (Ig) domain located at its N-terminus. The presence of Ig domains makes CD22 a member of the immunoglobulin superfamily. CD22 functions as an inhibitory receptor for B cell receptor (BCR) signalling.

Increased expression of CD22 is seen in non-Hodgkin and other lymphomas. Various monoclonal antibodies targeting CD22 are known, including epratuzumab, inotuzumab ozogamicin, m971 and m972.

### Chimeric Antigen Receptors (CAR)

The cell of the present invention may also comprise one or more chimeric antigen receptor(s). The CAR(s) may be specific for a tumour-associated antigen. Alternatively the CAR may comprise an autoantigen, or part thereof, in its extracellular domain, so that it binds autoantibodies produced by pathogenic B cells.

Classical CARs are chimeric type I trans-membrane proteins which connect an extracellular antigen-recognizing domain (binder) to an intracellular signalling domain (endodomain). The binder is typically a single-chain variable fragment (scFv) derived from a monoclonal antibody (mAb), but it can be based on other formats which comprise an antibody-like or ligand-based antigen binding site. A trans-membrane domain anchors the protein in the cell membrane and connects the spacer to the endodomain.

Early CAR designs had endodomains derived from the intracellular parts of either the γ chain of the FcεR1 or CD3ζ. Consequently, these first generation receptors transmitted immunological signal 1, which was sufficient to trigger T-cell killing of cognate target cells but failed to fully activate the T-cell to proliferate and survive. To overcome this limitation, compound endodomains have been constructed: fusion of the intracellular part of a T-cell co-stimulatory molecule to that of CD3ζ results in second generation receptors which can transmit an activating and co-stimulatory signal simultaneously after antigen recognition. The co-stimulatory domain most commonly used is that of CD28. This supplies the most potent co-stimulatory signal—namely immunological signal 2, which triggers T-cell proliferation. Some receptors have also been described which include TNF receptor family endodomains, such as the closely related OX40 and 41BB which transmit survival signals. Even more potent third generation CARs have now been described which have endodomains capable of transmitting activation, proliferation and survival signals.

CAR-encoding nucleic acids may be transferred to T cells using, for example, retroviral vectors. In this way, a large number of antigen-specific T cells can be generated for adoptive cell transfer. When the CAR binds the target-antigen, this results in the transmission of an activating signal to the T-cell it is expressed on. Thus the CAR directs the specificity and cytotoxicity of the T cell towards cells expressing the targeted antigen.

The cell of the present invention may comprise one or more CAR(s).

The CAR(s) may comprise an antigen-binding domain, a spacer domain, a transmembrane domain and an endodomain. The endodomain may comprise or associate with a domain which transmit T-cell activation signals.

### CAR Antigen Binding Domain

The antigen-binding domain is the portion of a CAR which recognizes antigen.

Numerous antigen-binding domains are known in the art, including those based on the antigen binding site of an antibody, antibody mimetics, and T-cell receptors. For example, the antigen-binding domain may comprise: a single-chain variable fragment (scFv) derived from a monoclonal antibody; a natural ligand of the target antigen; a peptide with sufficient affinity for the target; a single domain binder such as a camelid; an artificial binder single as a Darpin; or a single-chain derived from a T-cell receptor.

### Cell Surface Antigen

The CAR may recognise a cell-surface antigen, i.e. an entity, such as a transmembrane protein which is expressed on the surface of a target cell, such as a tumour cell.

The CAR may specifically bind a tumour-associated cell-surface antigen.

Various tumour associated antigens (TAA) are known, some of which are shown in Table 2. The antigen-binding domain used in the present invention may be a domain which is capable of binding a TAA as indicated therein.

TABLE 2

| Cancer type | TAA |
|---|---|
| Diffuse Large B-cell Lymphoma | CD19, CD20, CD22 |
| Breast cancer | ErbB2, MUC1 |
| AML | CD13, CD33 |
| Neuroblastoma | GD2, NCAM, ALK, GD2 |
| B-CLL | CD19, CD52, CD160 |
| Colorectal cancer | Folate binding protein, CA-125 |
| Chronic Lymphocytic Leukaemia | CD5, CD19 |
| Glioma | EGFR, Vimentin |
| Multiple myeloma | BCMA, CD138 |
| Renal Cell Carcinoma | Carbonic anhydrase IX, G250 |
| Prostate cancer | PSMA |
| Bowel cancer | A33 |

Where the CAR recognises a B-cell lymphoma or leukemia antigen (such as CD19, CD20, CD52, CD160 or CD5), the CCR may recognise another B-cell antigen, such as CD22.

### Prostate-Cancer Associated Antigens

The CAR may specifically bind a cell-surface antigen associated with prostate cancer, such as prostate stem cell antigen (PSCA) or prostate-specific membrane antigen (PSMA).

PSCA is a glycosylphosphatidylinositol-anchored cell membrane glycoprotein. It is is up-regulated in a large proportion of prostate cancers and is also detected in cancers of the bladder and pancreas.

Various anti-PSCA antibodies are known, such as 7F5 (Morgenroth et al (Prostate (2007) 67:1121-1131); 1G8 (Hillerdal et al (2014) BMC Cancer 14:30); and Ha1-4.117 (Abate-Daga et al (2014) 25:1003-1012).

The CCR-expressing cell of the invention may also express an anti-PSCA CAR which may comprise an antigen binding domain based on one of these antibodies.

PSMA is is a zinc metalloenzyme that resides in membranes. PSMA is strongly expressed in the human prostate, being a hundredfold greater than the expression in most other tissues. In cancer, it is upregulated in expression and has been called the second-most-upregulated gene in prostate cancer, with increase of 8- to 12-fold over the noncancerous prostate. In addition to the expression in the human prostate and prostate cancer, PSMA is also found to be highly expressed in tumor neovasculature but not normal vasculature of all types of solid tumors, such as kidney, breast, colon, etc.

Various anti-PSMA antibodies are known, such as 7E11, J591, J415, and Hybritech PEQ226.5 and PM2J004.5 each of which binds a distinct epitope of PSMA (Chang et al (1999) Cancer Res 15:3192-8).

The CCR-expressing cell of the invention may also express an anti-PSMA CAR which may comprise an antigen binding domain based on one of these antibodies.

For example, the CCR may comprise an scFv based on J591, having the sequence shown as SEQ ID No. 35.

(J591 scFv)
SEQ ID No. 35
EVQLQQSGPELKKPGTSVRISCKTSGYTFTEYTIHWVKQSHGKSLEWIG
NINPNNGGTTYNQKFEDKATLTVDKSSSTAYMELRSLTSEDSAVYYCAA
GWNFDYWGQGTTLTVSSGGGGSGGGGSGGGGSDIVMTQSHKFMSTSVGD
RVSIICKASQDVGTAVDWYQQKPGQSPKLLIYWASTRHTGVPDRFTGSG
SGTDFTLTITNVQSEDLADYFCQQYNSYPLTFGAGTMLDLKR

### Autoantibody Receptors

An autoimmune disease is a condition arising from an abnormal immune response to a functioning body part. Autoimmunity is the presence of self-reactive immune response (e.g., auto-antibodies, self-reactive T cells), with or without damage or pathology resulting from it. Autoantibodies are a hallmark of most autoimmune disorders.

A CAR-based approach can be used to treat autoimmune diseases and other disorders associated with an abnormal or undesirable immune response. In order to identify and kill pathogenic B cells associated with autoimmune diseases, the CAR extracellular domain may bind one or more autoantibodies. The extracellular domain may, for example, comprise an autoantibody binding entity such as an anti-idiotype antibody, or it may simply comprise the autoantigen itself, or part thereof.

Autoimmune disorders include Celiac disease, Diabetes mellitus type 1, Graves' disease, Inflammatory bowel disease, Multiple sclerosis (MS), Alopecia areata, Addison's disease, Pernicious anemia, psoriasis, Rheumatoid arthritis (RA), Systemic lupus erythematosus (SLE), Mucosal pemphigus vulgaris (mPV), Mucocutaneous pemphigus vulgaris (mcPV), Membranous nephropathy (MN) and myasthenia gravis (MG).

The extracellular domain of the CAR may contain all or part of an autoantigen, for example: cell adhesion protein desmoglein 3 (DSG3) or DSG1; Muscle-specific receptor tyrosine kinase (MuSK); Acetylcholine receptor (AChR); Phosphlipase A2 receptor (PLA2R) or Aquaporin-4 (AQP4).

Other undesirable immune responses include allergies, host vs graft responses and immune responses against therapies, such as Hemophilia A patients who develop antibodies against Factor VIII. In this latter example, using a CAR which comprises part or all of FVIII as its extracellular domain can destroy the antibodies and B cells that neutralise the FVIII clotting factor.

### CAR Transmembrane Domain

The transmembrane domain is the sequence of a CAR that spans the membrane. It may comprise a hydrophobic alpha helix. The CAR transmembrane domain may be derived from CD28, which gives good receptor stability.

### CAR Signal Peptide

The CAR and CCR described herein may comprise a signal peptide so that when it/they is expressed in a cell, such as a T-cell, the nascent protein is directed to the endoplasmic reticulum and subsequently to the cell surface, where it is expressed.

The core of the signal peptide may contain a long stretch of hydrophobic amino acids that has a tendency to form a single alpha-helix. The signal peptide may begin with a short positively charged stretch of amino acids, which helps to enforce proper topology of the polypeptide during translocation. At the end of the signal peptide there is typically a stretch of amino acids that is recognized and cleaved by signal peptidase. Signal peptidase may cleave either during or after completion of translocation to generate a free signal peptide and a mature protein. The free signal peptides are then digested by specific proteases.

The signal peptide may be at the amino terminus of the molecule.

The signal peptide may comprise the sequence shown as SEQ ID No. 5 or 11 above.

### CAR Endodomain

The endodomain is the signal-transmission portion of the CAR. It may be part of or associate with the intracellular domain of the CAR. After antigen recognition, receptors cluster, native CD45 and CD148 are excluded from the synapse and a signal is transmitted to the cell. The most commonly used endodomain component is that of CD3-zeta which contains 3 ITAMs. This transmits an activation signal to the T cell after antigen is bound. CD3-zeta may not provide a fully competent activation signal and additional co-stimulatory signalling may be needed. Co-stimulatory signals promote T-cell proliferation and survival. There are two main types of co-stimulatory signals: those that belong the Ig family (CD28, ICOS) and the TNF family (OX40, 41BB, CD27, GITR etc). For example, chimeric CD28 and OX40 can be used with CD3-Zeta to transmit a proliferative/survival signal, or all three can be used together.

The endodomain may comprise:
(i) an ITAM-containing endodomain, such as the endodomain from CD3 zeta; and/or
(ii) a co-stimulatory domain, such as the endodomain from CD28 or ICOS; and/or
(iii) a domain which transmits a survival signal, for example a TNF receptor family endodomain such as OX-40, 4-1 BB, CD27 or GITR.

A number of systems have been described in which the antigen recognition portion is on a separate molecule from the signal transmission portion, such as those described in WO015/150771; WO2016/124930 and WO2016/030691. The CAR of the present invention may therefore comprise an antigen-binding component comprising an antigen-binding domain and a transmembrane domain; which is capable of interacting with a separate intracellular signalling component comprising a signalling domain. The vector of the invention may express a CAR signalling system comprising such an antigen-binding component and intracellular signalling component.

### Nucleic Acid Sequences/Constructs

The present invention also provides nucleic acid sequences and nucleic acid constructs encoding a CCR of the invention.

The invention provides a nucleic acid sequence for producing a chimeric cytokine receptor as illustrated schematically in FIG. 1A. As explained above it is only necessary to encode one of the two chains of this CCR by recombinant means, as it will associate with the complementary endogenous chain inside the cell. The nucleic acid sequence encodes a polypeptide comprising interleukin-18 receptor 1 (IL18R1) or interleukin-18 receptor accessory protein (IL18RAP) tethered to an IL-18 molecule and may have the following general structure:
IL18-L-IL18R1ecto-TM-IL18R1endo or
IL18-L-IL18RAPecto-TM-IL18RAPendo
in which:
IL18 is a nucleic acid sequence encoding an IL18 molecule with a truncated or non-functional signal peptide;
L is a nucleic acid sequence encoding a flexible linker;
IL18R1ecto is a nucleic acid sequence encoding the interleukin-18 receptor 1 (IL18R1) ectodomain
TM is a nucleic acid sequence encoding a transmembrane domain
IL18R1endo is a nucleic acid sequence encoding IL18R1 endodomain
IL18RAPecto is a nucleic acid sequence encoding the ectodomain of interleukin-18 receptor accessory protein (IL18RAP);
IL18RAPendo is a nucleic acid sequence encoding the endodomain of IL18RAP.

A nucleic acid construct of the invention may comprise a first nucleic acid sequence encoding the first polypeptide; and a second nucleic acid sequence encoding the second polypeptide.

The nucleic acid construct may have the following structure:
ecto1-spacer1-TM1-IL18R1endo-coexpr-ecto2-spacer2-TM2-IL18RAPendo, or
ecto1-spacer1-TM1-IL18RAPendo-coexpr-ecto2-spacer2-TM2-IL18R1endo
in which
ecto1 is a nucleic acid sequence encoding the ectodomain of the first polypeptide;
spacer 1 is a nucleic acid sequence encoding the spacer of the first polypeptide;
TM1 is a a nucleic acid sequence encoding the transmembrane domain of the first polypeptide;
IL18R1endo is a nucleic acid sequence encoding the interleukin-18 receptor 1 (IL18R1) endodomain;
coexpr is a nucleic acid sequence enabling co-expression of both polypeptides as separate entities
ecto2 is a nucleic acid sequence encoding the ectodomain of the second polypeptide;
spacer 2 is a nucleic acid sequence encoding the spacer of the second polypeptide;
TM2 is a a nucleic acid sequence encoding the transmembrane domain of the second polypeptide;
IL18RAPendo is a nucleic acid sequence encoding the endodomain of interleukin-18 receptor accessory protein (IL18RAP).

The ectodomain of the first and second polypeptides may be capable of dimerising spontaneously to produce a constitutively active CCR. Endo1 and endo2 may, for example, encode a heavy chain constant domain (HC) and a light chain constant domain (LC) in either order; or two domains capable of forming a coiled coil structure, such as a leucine zipper.

When the nucleic acid construct is expressed in a cell, such as a T-cell, it encodes a polypeptide which is cleaved at the cleavage site such that the first and second polypeptides are co-expressed at the cell surface.

The present invention also provides a nucleic acid construct encoding a first and a second polypeptides of the invention and a CAR. Such a construct may have the structure:
(i) CCRecto1-CCRspacer1-CCRTM1-CCRendo1-coexpr1-CCRecto2-CCRspacer2-CCRTM2-CCRendo2-coexpr2-CARAgB-CARspacer-CARTM-CARendo;
(ii) CCRecto1-CCRspacer1-CCRTM1-CCRendo1-coexpr1-CARAgB-CARspacer-CARTM-CARendo-coexpr2-CCRecto2-CCRspacer2-CCRTM2-CCRendo2; or
(iii) CARAgB-CARspacer-CARTM-CARendo-coexpr1-CCRecto1-CCRspacer1-CCRTM1-CCRendo1-coexpr2-CCRecto2-CCRspacer2-CCRTM2-CCRendo2;
in which
CCRecto1 is a nucleic acid sequence encoding the ectodomain of the first polypeptide of the CCR;
CCRspacer1 is a nucleic acid sequence encoding the spacer of the first polypeptide of the CCR;
CCRTM1 is a nucleic acid sequence encoding the transmembrane domain of the first polypeptide of the CCR;
CCRendo1 is a nucleic acid sequence encoding the endodomain of the first polypeptide of the CCR;
CCRecto2 is a nucleic acid sequence encoding the ectodomain of the second polypeptide of the CCR;
CCRspacer2 is a nucleic acid sequence encoding the spacer of the second polypeptide of the CCR;
CCRTM2 is a nucleic acid sequence encoding the transmembrane domain of the second polypeptide of the CCR;
CCRendo2 is a nucleic acid sequence encoding the endodomain of the second polypeptide of the CCR;
Coexpr1 and coexpr2 are nucleic acid sequences which may be the same or different, which enable co-expression of the two flanking sequences;
CARAgB is a nucleic acid sequence encoding the antigen-binding domain of the CAR;
CARspacer is a nucleic acid sequence encoding the spacer of the CAR;
CARTM is a nucleic acid sequence encoding the transmembrane domain of the CAR; and
CARendo is a nucleic acid sequence encoding the endodomain of the CAR.

As used herein, the terms “polynucleotide”, “nucleotide”, and “nucleic acid” are intended to be synonymous with each other.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described here to reflect the codon usage of any particular host organism in which the polypeptides are to be expressed.

Nucleic acids according to the invention may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the use as described herein, it is to be understood that the polynucleotides may be modified by any method available in the art. Such modifications may be carried out in order to enhance the in vivo activity or life span of polynucleotides of interest.

The terms “variant”, “homologue” or “derivative” in relation to a nucleotide sequence include any substitution of, variation of, modification of, replacement of, deletion of or addition of one (or more) nucleic acid from or to the sequence.

In the structure above, “coexpr” is a nucleic acid sequence enabling co-expression of both first and second CARs. It may be a sequence encoding a cleavage site, such that the nucleic acid construct produces comprises two or more CCRs, or a CCR and a CAR, joined by a cleavage site(s). The cleavage site may be self-cleaving, such that when the polypeptide is produced, it is immediately cleaved into individual peptides without the need for any external cleavage activity.

The cleavage site may be any sequence which enables the first and second polypeptides of the CCR; or the CCR and CAR, to become separated.

The term “cleavage” is used herein for convenience, but the cleavage site may cause the peptides to separate into individual entities by a mechanism other than classical cleavage. For example, for the Foot-and-Mouth disease virus (FMDV) 2A self-cleaving peptide (see below), various models have been proposed for to account for the “cleavage” activity: proteolysis by a host-cell proteinase, autoproteolysis or a translational effect (Donnelly et al (2001) J. Gen. Virol. 82:1027-1041). The exact mechanism of such “cleavage” is not important for the purposes of the present invention, as long as the cleavage site, when positioned between nucleic acid sequences which encode proteins, causes the proteins to be expressed as separate entities.

The cleavage site may be a furin cleavage site.

Furin is an enzyme which belongs to the subtilisin-like proprotein convertase family. The members of this family are proprotein convertases that process latent precursor proteins into their biologically active products. Furin is a calcium-dependent serine endoprotease that can efficiently cleave precursor proteins at their paired basic amino acid processing sites. Examples of furin substrates include proparathyroid hormone, transforming growth factor beta 1 precursor, proalbumin, pro-beta-secretase, membrane type-1 matrix metalloproteinase, beta subunit of pro-nerve growth factor and von Willebrand factor. Furin cleaves proteins just downstream of a basic amino acid target sequence (canonically, Arg-X-(Arg/Lys)-Arg′) and is enriched in the Golgi apparatus.

The cleavage site may be a Tobacco Etch Virus (TEV) cleavage site.

TEV protease is a highly sequence-specific cysteine protease which is chymotrypsin-like proteases. It is very specific for its target cleavage site and is therefore frequently used for the controlled cleavage of fusion proteins both in vitro and in vivo. The consensus TEV cleavage site is ENLYFQ\S (where ‘\’ denotes the cleaved peptide bond). Mammalian cells, such as human cells, do not express TEV protease. Thus in embodiments in which the present nucleic acid construct comprises a TEV cleavage site and is expressed in a mammalian cell—exogenous TEV protease must also expressed in the mammalian cell.

The cleavage site may encode a self-cleaving peptide.

A ‘self-cleaving peptide’ refers to a peptide which functions such that when the polypeptide comprising the proteins and the self-cleaving peptide is produced, it is immediately “cleaved” or separated into distinct and discrete first and second polypeptides without the need for any external cleavage activity.

The self-cleaving peptide may be a 2A self-cleaving peptide from an aphtho- or a cardiovirus. The primary 2A/2B cleavage of the aptho- and cardioviruses is mediated by 2A “cleaving” at its own C-terminus. In apthoviruses, such as foot-and-mouth disease viruses (FMDV) and equine rhinitis A virus, the 2A region is a short section of about 18 amino acids, which, together with the N-terminal residue of protein 2B (a conserved proline residue) represents an autonomous element capable of mediating “cleavage” at its own C-terminus (Donelly et al (2001) as above).

“2A-like” sequences have been found in picornaviruses other than aptho- or cardioviruses, ‘picornavirus-like’ insect viruses, type C rotaviruses and repeated sequences within Trypanosoma spp and a bacterial sequence (Donnelly et al (2001) as above). The nucleic acid construct of the invention may encode one or these 2A-like cleavage sequences. A suitable sequence is given above (SEQ ID No. 10)

### Vector

The present invention also provides a vector, or kit of vectors, which comprises one or more nucleic acid sequence(s) encoding a CCR according to the invention and optionally one or more CAR(s). Such a vector may be used to introduce the nucleic acid sequence(s) into a host cell so that it expresses a CCR according to the invention.

The vector may, for example, be a plasmid or a viral vector, such as a retroviral vector or a lentiviral vector, or a transposon based vector or synthetic mRNA.

The vector may be capable of transfecting or transducing a cell, such as T cell or a NK cell.

### Cell

The present invention provides a cell which comprises a CCR of the invention and optionally one of more CAR(s).

The cell may comprise a nucleic acid or a vector of the present invention.

The cell may be a cytolytic immune cell such as a T cell or an NK cell.

T cells or T lymphocytes are a type of lymphocyte that play a central role in cell-mediated immunity. They can be distinguished from other lymphocytes, such as B cells and natural killer cells (NK cells), by the presence of a T-cell receptor (TCR) on the cell surface. There are various types of T cell, as summarised below.

Helper T helper cells (TH cells) assist other white blood cells in immunologic processes, including maturation of B cells into plasma cells and memory B cells, and activation of cytotoxic T cells and macrophages. TH cells express CD4 on their surface. TH cells become activated when they are presented with peptide antigens by MHC class II molecules on the surface of antigen presenting cells (APCs). These cells can differentiate into one of several subtypes, including TH1, TH2, TH3, TH17, Th9, or TFH, which secrete different cytokines to facilitate different types of immune responses.

Cytolytic T cells (TC cells, or CTLs) destroy virally infected cells and tumor cells, and are also implicated in transplant rejection. CTLs express the CD8 at their surface. These cells recognize their targets by binding to antigen associated with MHC class I, which is present on the surface of all nucleated cells. Through IL-10, adenosine and other molecules secreted by regulatory T cells, the CD8+ cells can be inactivated to an anergic state, which prevent autoimmune diseases such as experimental autoimmune encephalomyelitis.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory T cells comprise three subtypes: central memory T cells (TCM cells) and two types of effector memory T cells (TEM cells and TEMRA cells). Memory cells may be either CD4+ or CD8+. Memory T cells typically express the cell surface protein CD45RO.

Regulatory T cells (Treg cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus.

Two major classes of CD4+ Treg cells have been described—naturally occurring Treg cells and adaptive Treg cells.

Naturally occurring Treg cells (also known as CD4+CD25+FoxP3+ Treg cells) arise in the thymus and have been linked to interactions between developing T cells with both myeloid (CD11c+) and plasmacytoid (CD123+) dendritic cells that have been activated with TSLP. Naturally occurring Treg cells can be distinguished from other T cells by the presence of an intracellular molecule called FoxP3. Mutations of the FOXP3 gene can prevent regulatory T cell development, causing the fatal autoimmune disease IPEX.

Adaptive Treg cells (also known as Tr cells or Th3 cells) may originate during a normal immune response.

The cell may be a Natural Killer cell (or NK cell). NK cells form part of the innate immune system. NK cells provide rapid responses to innate signals from virally infected cells in an MHC independent manner

NK cells (belonging to the group of innate lymphoid cells) are defined as large granular lymphocytes (LGL) and constitute the third kind of cells differentiated from the common lymphoid progenitor generating B and T lymphocytes. NK cells are known to differentiate and mature in the bone marrow, lymph node, spleen, tonsils and thymus where they then enter into the circulation.

The CCR-expressing cells of the invention may be any of the cell types mentioned above.

T or NK cells according to the first aspect of the invention may either be created ex vivo either from a patient's own peripheral blood (1st party), or in the setting of a haematopoietic stem cell transplant from donor peripheral blood (2nd party), or peripheral blood from an unconnected donor (3rd party).

Alternatively, T or NK cells according to the first aspect of the invention may be derived from ex vivo differentiation of inducible progenitor cells or embryonic progenitor cells to T or NK cells. Alternatively, an immortalized T-cell line which retains its lytic function and could act as a therapeutic may be used.

In all these embodiments, CCR-expressing cells are generated by introducing DNA or RNA coding for the or each CCR(s) by one of many means including transduction with a viral vector, transfection with DNA or RNA.

The cell of the invention may be an ex vivo T or NK cell from a subject. The T or NK cell may be from a peripheral blood mononuclear cell (PBMC) sample. T or NK cells may be activated and/or expanded prior to being transduced with nucleic acid encoding the molecules providing the CCR according to the first aspect of the invention, for example by treatment with an anti-CD3 monoclonal antibody.

The T or NK cell of the invention may be made by:
(i) isolation of a T or NK cell-containing sample from a subject or other sources listed above; and
(ii) transduction or transfection of the T or NK cells with one or more a nucleic acid sequence(s) encoding a CCR.

The T or NK cells may then by purified, for example, selected on the basis of expression of the antigen-binding domain of the antigen-binding polypeptide.

### Pharmaceutical Composition

The present invention also relates to a pharmaceutical composition containing a plurality of cells according to the invention.

The pharmaceutical composition may additionally comprise a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

### Method of Treatment

The present invention provides a method for treating and/or preventing a disease which comprises the step of administering the cells of the present invention (for example in a pharmaceutical composition as described above) to a subject.

A method for treating a disease relates to the therapeutic use of the cells of the present invention. The cells may be administered to a subject having an existing disease or condition in order to lessen, reduce or improve at least one symptom associated with the disease and/or to slow down, reduce or block the progression of the disease.

The method for preventing a disease relates to the prophylactic use of the cells of the present invention. Such cells may be administered to a subject who has not yet contracted the disease and/or who is not showing any symptoms of the disease to prevent or impair the cause of the disease or to reduce or prevent development of at least one symptom associated with the disease. The subject may have a predisposition for, or be thought to be at risk of developing, the disease.

The method may involve the steps of:
(i) isolating a T or NK cell-containing sample;
(ii) transducing or transfecting such cells with a nucleic acid sequence or vector provided by the present invention;
(iii) administering the cells from (ii) to a subject.

The T or NK cell-containing sample may be isolated from a subject or from other sources, for example as described above. The T or NK cells may be isolated from a subject's own peripheral blood (1st party), or in the setting of a haematopoietic stem cell transplant from donor peripheral blood (2nd party), or peripheral blood from an unconnected donor (3rd party).

The present invention provides a CCR-expressing cell of the present invention for use in treating and/or preventing a disease.

The invention also relates to the use of a CCR-expressing cell of the present invention in the manufacture of a medicament for the treatment and/or prevention of a disease.

The disease to be treated and/or prevented by the methods of the present invention may be a cancerous disease, such as bladder cancer, breast cancer, colon cancer, endometrial cancer, kidney cancer (renal cell), leukaemia, lung cancer, melanoma, non-Hodgkin lymphoma, pancreatic cancer, prostate cancer and thyroid cancer.

In particular the cancer may be a solid cancer. The cancer may produce a so-called “cold tumour” i.e. a tumour that is not likely to trigger a strong immune response. Cold tumours tend to be surrounded by cells that are able to suppress T cells from attacking the tumour cells and killing them. Cold tumours have low immune infiltrates and usually do not respond to immunotherapy. Examples of cold tumours include cancers of the colon, prostate, ovary and pancreas, neuroblastoma and glioblastoma.

The cancer may be a cancer with relatively low levels of neo-antigen. Neoantigens are tumour-specific antigens generated by nonsynonymous mutations that occur during cellular transformation. Because they are foreign to the host genome, they are not subject to central tolerance and can be highly immunogenic. Examples of cancers with low levels of neo-antigen include: prostate, pancreatic and ovarian cancer, neuroblastoma, renal carcinoma and merkel cell carcinoma.

Where CCR is activated by the ligand PSA, the cancer may be prostate cancer.

The cells of the present invention may be capable of killing target cells, such as cancer cells. The target cell may be characterised by the presence of a tumour secreted ligand or chemokine ligand in the vicinity of the target cell. The target cell may be characterised by the presence of a soluble ligand together with the expression of a tumour-associated antigen (TAA) at the target cell surface.

The disease to be treated and/or prevented by the methods of the present invention may be an autoimmune disease, allergy, or other condition associated with an inappropriate or undesirable immune response.

Autoimmune disorders include Celiac disease, Diabetes mellitus type 1, Graves' disease, Inflammatory bowel disease, Multiple sclerosis (MS), Alopecia areata, Addison's disease, Pernicious anemia, psoriasis, Rheumatoid arthritis (RA), Systemic lupus erythematosus (SLE), Mucosal pemphigus vulgaris (mPV), Mucocutaneous pemphigus vulgaris (mcPV), Membranous nephropathy (MN) and myasthenia gravis (MG).

The cells of the present invention may be capable of cells with produce self-reactive or other undesirable antibodies (such as antibodies against a transplant or treatment). The cells of the invention may be capable of killing autoantibody-producing B cells. The target cell may be characterised by expression of and/or capacity to secrete undesirable antibodies such as autoantibodies.

The cells and pharmaceutical compositions of present invention may be for use in the treatment and/or prevention of the diseases described above.

The cells and pharmaceutical compositions of present invention may be for use in any of the methods described above.

The invention will now be further described by way of Examples, which are meant to serve to assist one of ordinary skill in the art in carrying out the invention and are not intended in any way to limit the scope of the invention.

### EXAMPLES

### Example 1—Investigating the Effect of Co-Expression of a CAR with an IL-18 CCR In Vitro

A constitutively active IL-18-signalling chimeric cytokine receptor was produced by linking IL-18 receptor endodomains to a “Fab” type exodomain (see the embodiment schematically illustrated in FIG. 1E). This structure uses the natural dimerization components of antibodies, namely the dimerization domain from the heavy and light chain constant regions. The chimeric cytokine receptor has two chains; a first polypeptide which comprises the antibody light K chain (shown as SEQ ID No. 3) and the IL18R1 TM domain (SEQ ID No. 8) and endodomain (SEQ ID No 9); and a second polypeptide which comprises the antibody heavy chain CH1 (SEQ ID No. 4) and the IL18RAP TM domain (SEQ ID No. 12) and endodomain (shown as SEQ ID No. 13).

Nucleic acid sequences encoding these two polypeptides were cloned in frame separated by a 2A-peptide encoding sequence.

Vectors were constructed as illustrated schematically in FIG. 2A. Both vectors expressed: a) the sort-suicide gene RQR8 which is described in WO2013/153391; and b) a second generation CAR having the GD2-binding domain described in WO2015/132604 and a 41BB-CD3z endodomain. One vector also expressed the IL-18 CCR.

T cells were transduced with these constructs and then co-cultured with either wild-type SupT1 cells (which do not express GD2) or SupT1 target cells transduced to express GD2. Briefly, 12.5×10⁴ transduced CAR T cells were co-cultured with 5×10⁴ target cells giving a E:T ratio of 1:4. Target cell killing was analysed by FACS and release of IL2 and IFNγ into the supernatant was analysed by ELISA. The results are shown in FIG. 2B. Co-expression of the IL-18 CCR maintained target cell killing, but improved cytokine release compared to cells expressing CAR alone.

### Example 2—Comparing the Effect of Coexpression of an IL2, IL7 or IL18 CCR In Vivo in a CT26-GD2+ Colon Carcinoma Animal Model

An in vivo experiment was conducted to evaluate the engraftment and anti-tumour activity of murine T cells co-expressing a CAR and CCR in an established colon carcinoma immunocompetent model in Balb/c mice.

The experimental design is illustrated schematically in FIG. 3. Briefly, 6-8 Weeks old Balb/c female mice were subcutaneously injected with 1×10⁶ CT26 colon carcinoma cells modified to express GD2. After tumour engraftment for 9 days, the mice were sub-lethally irritated with 5 Gy Total Body Irradiation (TBI). Murine CAR-T cells were made by transducing cells with either: a single vector expressing a GD2 CAR (GD2 CAR); or a bi-cistronic vector co-expressing GD2 CAR and one of an IL2-CCR, an IL-7 CCR or an IL-18 CCR. One day after TBI, 1×10⁶ transduced CAR T cells were administered by intravenous injection. Tumour growth was monitored twice a week via calliper. Peripheral blood was taken every 7 days and analysed for the presence of transduced CAR T cells via the markers CD3 and Thy1.1. The CD3 marker was used to identify the lymphocyte population within the total CD45+ immune cells. THY1.1 was used to identify the transduced CD3 T cells within the total T cell population. Mice were euthanised when the tumour reached a maximum length or breadth of 1 cm³ or mouse with sudden body weight loss 20%. Spleen and tumour were collected at the time of euthanisation for CAR T cell tracking.

The results are shown in FIG. 4 (tumour volume) and FIG. 5 (peripheral blood T cell count).

The CT26 GD2⁺ tumour was spared by cells expressing the GD2 CAR alone or cells co-expressing the GD2 CAR with an IL2-CCR or IL7-CCR. By contrast, the CAR T cells transduced with the bi-cistronic vector expressing the IL18-CCR exhibited potent anti-tumour activity and extended the survival of the Balb/c mice with established tumour burden, with undetectable tumour up to day 50 in 4 out of 8 mice.

The enhanced tumour control by the IL18-CCR GD2 CAR T cells was also accompanied by a higher engraftment of the CAR T cells. The IL18 CCR CAR T cells displayed the highest degree of engraftment with a peak at day 10 post infusion and sustained high peripheral persistence of the transduced CAR T cells until the end of the experiment.

The Kaplan Meyer survival curve (FIG. 6) demonstrated a degree of survival improvement in animals which received CAR-T cells expressing IL2 or IL7 CCR, but this was greatly increased in animals which received CAR-T cells expressing IL18 CCR: 50% of the mice in the IL18 CCR transduced cohort were alive by day 50 post CAR T cell injection.

### Example 3—Investigating Differences in Gene Expression in Activated and Non-Activated CAR-T Cells Expressing IL7- or IL-18 CCR

Expression of a panel of 800 genes was analysed for activated and non-activated CAR-T cells expressing IL7- or IL-18 CCR. The results of Principal Component Analysis (PCA) are shown in FIG. 7. Clear differences were observed between CAR-T cells expressing IL7- or IL-18 CCR in both the non-activated and activated states.

In order to investigate this further, changes in expression for individual genes were analysed for IL18 CCR-expressing cells in both an activated and non-activated state, using IL7 CCR as baseline for the differential gene expression calculation.

The results are shown in FIG. 8. The expression of some genes, notably the genes encoding chemokines CCL22, CCL17, CXCL8 and the interleukin IL1A are significantly upregulated in CAR-T cells expressing IL-18 CCR compared to CAR-T cells expressing IL-7 CCR.

This observation suggests that the mechanism by which IL-7 and IL-18 CCRs support CAR-T function may fundamentally differ. Without wishing to be bound by theory, the inventors predict that the IL-18 CCR functions by interacting with the host immune system rather than by having a direct effect on the CAR-T cell itself. This is consistent with the fact that IL-18 as a cytokine does not directly induce T-cell proliferation, unlike IL-7. The differential expression of chemokines such as CCL22, CXCL8 and CCL17 suggests that the IL-18 CCR functions primarily by recruiting cells of the host immune system to the tumour site.

This mechanism is thought to underpin the observed results in the CT-26-GD2+ mouse model. This immunocompetent animal model can be thought of a cold tumour i.e. one which is likely to be immune compromised. Cold tumors tend to be surrounded by cells that are able to suppress T cells and they typically do not respond to immunotherapy. The immunocompetent animal model also has low levels of neo-antigen. Using this model, CAR-T cells expressing the IL-18 CCR were significantly better at controlling the tumour than CAR T-cells expressing an IL-7 CCR. The recruitment of host immune cells to the tumour site by CAR-T cells expressing an IL-18 CCR may be more important for tackling this colon carcinoma tumour model than boosting the activity of the CAR-T cell itself.

By contrast, a hot tumour which is likely to trigger a strong immune response, or a tumour with a higher level of neo-antigen present which could trigger neo-antigen driven tumour killing activity, is likely to be boosted by the proliferatory effect of, for example, an IL2- or IL7-CCR.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A chimeric cytokine receptor (CCR) which comprises two polypeptides:
(i) a first polypeptide which comprises:
(a) an ectodomain which comprises a first dimerization domain;
(b) an interleukin-18 receptor 1 (IL18R1) endodomain; and
(ii) a second polypeptide which comprises:
(a) an ectodomain which comprises a second dimerization domain which spontaneously dimerises with the first dimerization domain;
(b) an interleukin-18 receptor accessory protein (IL18RAP) endodomain;
wherein one of the first and second dimerization domains comprises a heavy chain constant domain (CH) and the other comprises a light chain constant domain (CL).

2-6. (canceled)

7. A cell which comprises a chimeric cytokine receptor according to claim 1.

8. A cell according to claim 7, which also comprises a chimeric antigen receptor.

9. A nucleic acid construct encoding a chimeric cytokine receptor (CCR) according to claim 1, which nucleic acid construct comprises a first nucleic acid sequence encoding the first polypeptide and a second nucleic acid sequence encoding the second polypeptide.

10. (canceled)

11. A nucleic acid construct according to claim 9 which also comprises a nucleic acid sequence which encodes a chimeric antigen receptor.

12. A vector comprising a nucleic acid construct according to claim 9.

13. A kit which comprises:
i) a vector comprising a nucleic acid sequence encoding a first polypeptide which comprises:
(a) an ectodomain which comprises a first dimerization domain;
(b) an interleukin-18 receptor 1 (IL18R1) endodomain; and
ii) a vector comprising a nucleic acid sequence encoding a second polypeptide which comprises:
(a) an ectodomain which comprises a second dimerization domain which spontaneously dimerises with the first dimerization domain;
(b) an interleukin-18 receptor accessory protein (IL18RAP) endodomain, wherein one of the first and second dimerization domains comprises a heavy chain contact domain (CH) and the other comprises a light chain contact domain (CL).

14. (canceled)

15. A method for making a cell which expresses a chimeric cytokine receptor (CCR), which method comprises the step of introducing: a nucleic acid construct according to claim 9, into a cell ex vivo.

16. A pharmaceutical composition comprising a plurality of cells according to claim 7.

17. (canceled)

18. A method for treating cancer or an autoimmune disease which comprises the step of administering a pharmaceutical composition according to claim 16 to a subject.

19. (canceled)

20. A vector comprising a nucleic acid construct according to claim 11.

21. A method for making a cell which co-expresses a chimeric cytokine receptor (CCR) and a chimeric antigen receptor (CAR), which comprises the step of introducing a nucleic acid construct according to claim 11 into a cell.

22. A pharmaceutical composition comprising a plurality of cells according to claim 8.

23. A method for treating cancer or an autoimmune disease which comprises the step of administering a pharmaceutical composition according to claim 22 to a subject.


SEQUENCE LISTING (62 sequences)

SEQ ID NO: 1 - protein, 541 aa, Homo sapiens
MNCRELPLTLWVLISVSTAESCTSRPHITVVEGEPFYLKHCSCSLAHEIETTTKSWYKSSGSQEHVELNPRSSSRIALHDCVLEFWPVELNDTGSYFFQMKNYTQKWKLNVIRRNKHSCFTERQVTSKIVEVKKFFQITCENSYYQTLVNSTSLYKNCKKLLLENNKNPTIKKNAEFEDQGYYSCVHFLHHNGKLFNITKTFNITIVEDRSNIVPVLLGPKLNHVAVELGKNVRLNCSALLNEEDVIYWMFGEENGSDPNIHEEKEMRIMTPEGKWHASKVLRIENIGESNLNVLYNCTVASTGGTDTKSFILVRKADMADIPGHVFTRGMIIAVLILVAVVCLVTVCVIYRVDLVLFYRHLTRRDETLTDGKTYDAFVSYLKECRPENGEEHTFAVEILPRVLEKHFGYKLCIFERDVVPGGAVVDEIHSLIEKSRRLIIVLSKSYMSNEVRYELESGLHEALVERKIKIILIEFTPVTDFTFLPQSLKLLKSHRVLKWKADKSLSYNSRFWKNLLYLMPAKTVKPGRDEPEVLPVLSES

SEQ ID NO: 2 - protein, 599 aa, Homo sapiens
MLCLGWIFLWLVAGERIKGFNISGCSTKKLLWTYSTRSEEEFVLFCDLPEPQKSHFCHRNRLSPKQVPEHLPFMGSNDLSDVQWYQQPSNGDPLEDIRKSYPHIIQDKCTLHFLTPGVNNSGSYICRPKMIKSPYDVACCVKMILEVKPQTNASCEYSASHKQDLLLGSTGSISCPSLSCQSDAQSPAVTWYKNGKLLSVERSNRIVVDEVYDYHQGTYVCDYTQSDTVSSWTVRAVVQVRTIVGDTKLKPDILDPVEDTLEVELGKPLTISCKARFGFERVFNPVIKWYIKDSDLEWEVSVPEAKSIKSTLKDEIIERNIILEKVTQRDLRRKFVCFVQNSIGNTTQSVQLKEKRGVVLLYILLGTIGTLVAVLAASALLYRHWIEIVLLYRTYQSKDQTLGDKKDFDAFVSYAKWSSFPSEATSSLSEEHLALSLFPDVLENKYGYSLCLLERDVAPGGVYAEDIVSIIKRSRRGIFILSPNYVNGPSIFELQAAVNLALDDQTLKLILIKFCYFQEPESLPHLVKKALRVLPTVTWRGLKSVPPNSRFWAKMRYHMPVKNSQGFTWNQLRITSRIFQWKGLSRTETTGRSSQPKEW

SEQ ID NO: 3 - protein, 107 aa, Homo sapiens [sequence omitted]

SEQ ID NO: 4 - protein, 97 aa, Homo sapiens
STKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 5 - protein, 19 aa, Synthetic construct
MGWSCIILFLVATATGVHS

SEQ ID NO: 6 - protein, 15 aa, Synthetic construct
EPKSCDKTHTCPPCP

SEQ ID NO: 7 - protein, 4 aa, Synthetic construct
KDPK

SEQ ID NO: 8 - protein, 21 aa, Synthetic construct
GMIIAVLILVAVVCLVTVCVI

SEQ ID NO: 9 - protein, 191 aa, Synthetic construct
YRVDLVLFYRHLTRRDETLTDGKTYDAFVSYLKECRPENGEEHTFAVEILPRVLEKHFGYKLCIFERDVVPGGAVVDEIHSLIEKSRRLIIVLSKSYMSNEVRYELESGLHEALVERKIKIILIEFTPVTDFTFLPQSLKLLKSHRVLKWKADKSLSYNSRFWKNLLYLMPAKTVKPGRDEPEVLPVLSES

SEQ ID NO: 10 - protein, 18 aa, Synthetic construct
EGRGSLLTCGDVEENPGP

SEQ ID NO: 11 - protein, 20 aa, Synthetic construct
METDTLILWVLLLLVPGSTG

SEQ ID NO: 12 - protein, 21 aa, Synthetic construct
GVVLLYILLGTIGTLVAVLAA

SEQ ID NO: 13 - protein, 220 aa, Synthetic construct
SALLYRHWIEIVLLYRTYQSKDQTLGDKKDFDAFVSYAKWSSFPSEATSSLSEEHLALSLFPDVLENKYGYSLCLLERDVAPGGVYAEDIVSIIKRSRRGIFILSPNYVNGPSIFELQAAVNLALDDQTLKLILIKFCYFQEPESLPHLVKKALRVLPTVTWRGLKSVPPNSRFWAKMRYHMPVKNSQGFTWNQLRITSRIFQWKGLSRTETTGRSSQPK

SEQ ID NO: 14 - protein, 193 aa, Homo sapiens
MAAEPVEDNCINFVAMKFIDNTLYFIAEDDENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 15 - protein, 157 aa, Synthetic construct
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 16 - protein, 26 aa, Synthetic construct
SGGGGSGGGGSGGGGSGGGGSGGGGS

SEQ ID NO: 17 - protein, 308 aa, Synthetic construct
CTSRPHITVVEGEPFYLKHCSCSLAHEIETTTKSWYKSSGSQEHVELNPRSSSRIALHDCVLEFWPVELNDTGSYFFQMKNYTQKWKLNVIRRNKHSCFTERQVTSKIVEVKKFFQITCENSYYQTLVNSTSLYKNCKKLLLENNKNPTIKKNAEFEDQGYYSCVHFLHHNGKLFNITKTFNITIVEDRSNIVPVLLGPKLNHVAVELGKNVRLNCSALLNEEDVIYWMFGEENGSDPNIHEEKEMRIMTPEGKWHASKVLRIENIGESNLNVLYNCTVASTGGTDTKSFILVRKADMADIPGHVFTR

SEQ ID NO: 18 - protein, 337 aa, Synthetic construct
FNISGCSTKKLLWTYSTRSEEEFVLFCDLPEPQKSHFCHRNRLSPKQVPEHLPFMGSNDLSDVQWYQQPSNGDPLEDIRKSYPHIIQDKCTLHFLTPGVNNSGSYICRPKMIKSPYDVACCVKMILEVKPQTNASCEYSASHKQDLLLGSTGSISCPSLSCQSDAQSPAVTWYKNGKLLSVERSNRIVVDEVYDYHQGTYVCDYTQSDTVSSWTVRAVVQVRTIVGDTKLKPDILDPVEDTLEVELGKPLTISCKARFGFERVFNPVIKWYIKDSDLEWEVSVPEAKSIKSTLKDEIIERNIILEKVTQRDLRRKFVCFVQNSIGNTTQSVQLKEKR

SEQ ID NO: 19 - protein, 16 aa, Synthetic construct
DSSGKPIPNPLLGLDS

SEQ ID NO: 20 - protein, 6 aa, Synthetic construct
SGGGGS

SEQ ID NO: 21 - protein, 113 aa, Synthetic construct
DIVMTQTAPSVFVTPGESVSISCRSSKSLLHSNGNTYLYWFLQRPGQSPQLLIYRMSNLASGVPDRFSGSGSGTDFTLRISRVEAEDVGVYYCMQHLEYPVTFGAGTKVEIKR

SEQ ID NO: 22 - protein, 16 aa, Synthetic construct
SGGGGSGGGGSGGGGS

SEQ ID NO: 23 - protein, 120 aa, Synthetic construct
QVQLQQSGPELVKPGASVKISCKVSGYAISSSWMNWVKQRPGQGLEWIGRIYPGDGDTKYNGKFKDKATLTVDKSSSTAYMQLSSLTSVDSAVYFCARDGYRYYFDYWGQGTSVTVSSDP

SEQ ID NO: 24 - protein, 46 aa, Synthetic construct
TTTPAPRPPTPAPTIASQPLSLRPEASRPAAGGAVHTRGLDFASDI

SEQ ID NO: 25 - protein, 9 aa, Synthetic construct
YPYDVPDYA

SEQ ID NO: 26 - protein, 116 aa, Synthetic construct
QVQLQQSGAELAKPGASVKMSCKTSGYSFSSYWMHWVKQRPGQGLEWIGYINPSTGYTENNQKFKDKVTLTADKSSNTAYMQLNSLTSEDSAVYYCARSGRLYFDVWGAGTTVTVS

SEQ ID NO: 27 - protein, 116 aa, Synthetic construct
DIVLTQSPPSLAVSLGQRATISCRASESIDLYGFTFMHWYQQKPGQPPKILIYRASNLESGIPARFSGSGSRTDFTLTINPVEADDVATYYCQQTHEDPYTFGGGTKLEIKRSDPA

SEQ ID NO: 28 - protein, 19 aa, Synthetic construct
EPKSPDKTHTSPPSPKDPK

SEQ ID NO: 29 - protein, 144 aa, Synthetic construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQ

SEQ ID NO: 30 - protein, 18 aa, Synthetic construct
ISLLPPLGVAISVIIIFY

SEQ ID NO: 31 - protein, 92 aa, Synthetic construct
LQCFCHLCTKDNFTCVTDGLCFVSVTETTDKVIHNSMCIAEIDLIPRDRPFVCAPSSKTGSVTTTYCCNQDHCNKIELPTTVKSSPGLGPVE

SEQ ID NO: 32 - protein, 22 aa, Synthetic construct
LAAVIAGPVCFVCISLMLMVYI

SEQ ID NO: 33 - protein, 128 aa, Synthetic construct
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSR

SEQ ID NO: 34 - protein, 114 aa, Synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRA

SEQ ID NO: 35 - protein, 238 aa, Synthetic construct
EVQLQQSGPELKKPGTSVRISCKTSGYTFTEYTIHWVKQSHGKSLEWIGNINPNNGGTTYNQKFEDKATLTVDKSSSTAYMELRSLTSEDSAVYYCAAGWNFDYWGQGTTLTVSSGGGGSGGGGSGGGGSDIVMTQSHKFMSTSVGDRVSIICKASQDVGTAVDWYQQKPGQSPKLLIYWASTRHTGVPDRFTGSGSGTDFTLTITNVQSEDLADYFCQQYNSYPLTFGAGTMLDLKR

SEQ ID NO: 36 - protein, 20 aa, Synthetic construct
YRVDLVLFYRHLTRRDETLT

SEQ ID NO: 37 - protein, 40 aa, Synthetic construct
YRVDLVLFYRHLTRRDETLTDGKTYDAFVSYLKECRPENG

SEQ ID NO: 38 - protein, 60 aa, Synthetic construct
YRVDLVLFYRHLTRRDETLTDGKTYDAFVSYLKECRPENGEEHTFAVEILPRVLEKHFGY

SEQ ID NO: 39 - protein, 80 aa, Synthetic construct
YRVDLVLFYRHLTRRDETLTDGKTYDAFVSYLKECRPENGEEHTFAVEILPRVLEKHFGYKLCIFERDVVPGGAVVDEIH

SEQ ID NO: 40 - protein, 100 aa, Synthetic construct
YRVDLVLFYRHLTRRDETLTDGKTYDAFVSYLKECRPENGEEHTFAVEILPRVLEKHFGYKLCIFERDVVPGGAVVDEIHSLIEKSRRLIIVLSKSYMSN

SEQ ID NO: 41 - protein, 120 aa, Synthetic construct
YRVDLVLFYRHLTRRDETLTDGKTYDAFVSYLKECRPENGEEHTFAVEILPRVLEKHFGYKLCIFERDVVPGGAVVDEIHSLIEKSRRLIIVLSKSYMSNEVRYELESGLHEALVERKIK

SEQ ID NO: 42 - protein, 140 aa, Synthetic construct
YRVDLVLFYRHLTRRDETLTDGKTYDAFVSYLKECRPENGEEHTFAVEILPRVLEKHFGYKLCIFERDVVPGGAVVDEIHSLIEKSRRLIIVLSKSYMSNEVRYELESGLHEALVERKIKIILIEFTPVTDFTFLPQSLK

SEQ ID NO: 43 - protein, 160 aa, Synthetic construct
YRVDLVLFYRHLTRRDETLTDGKTYDAFVSYLKECRPENGEEHTFAVEILPRVLEKHFGYKLCIFERDVVPGGAVVDEIHSLIEKSRRLIIVLSKSYMSNEVRYELESGLHEALVERKIKIILIEFTPVTDFTFLPQSLKLLKSHRVLKWKADKSLSYNS

SEQ ID NO: 44 - protein, 180 aa, Synthetic construct
YRVDLVLFYRHLTRRDETLTDGKTYDAFVSYLKECRPENGEEHTFAVEILPRVLEKHFGYKLCIFERDVVPGGAVVDEIHSLIEKSRRLIIVLSKSYMSNEVRYELESGLHEALVERKIKIILIEFTPVTDFTFLPQSLKLLKSHRVLKWKADKSLSYNSRFWKNLLYLMPAKTVKPGRD

SEQ ID NO: 45 - protein, 20 aa, Synthetic construct
SALLYRHWIEIVLLYRTYQS

SEQ ID NO: 46 - protein, 40 aa, Synthetic construct
SALLYRHWIEIVLLYRTYQSKDQTLGDKKDFDAFVSYAKW

SEQ ID NO: 47 - protein, 60 aa, Synthetic construct
SALLYRHWIEIVLLYRTYQSKDQTLGDKKDFDAFVSYAKWSSFPSEATSSLSEEHLALSL

SEQ ID NO: 48 - protein, 80 aa, Synthetic construct
SALLYRHWIEIVLLYRTYQSKDQTLGDKKDFDAFVSYAKWSSFPSEATSSLSEEHLALSLFPDVLENKYGYSLCLLERDV

SEQ ID NO: 49 - protein, 100 aa, Synthetic construct
SALLYRHWIEIVLLYRTYQSKDQTLGDKKDFDAFVSYAKWSSFPSEATSSLSEEHLALSLFPDVLENKYGYSLCLLERDVAPGGVYAEDIVSIIKRSRRG

SEQ ID NO: 50 - protein, 120 aa, Synthetic construct
SALLYRHWIEIVLLYRTYQSKDQTLGDKKDFDAFVSYAKWSSFPSEATSSLSEEHLALSLFPDVLENKYGYSLCLLERDVAPGGVYAEDIVSIIKRSRRGIFILSPNYVNGPSIFELQAA

SEQ ID NO: 51 - protein, 140 aa, Synthetic construct
SALLYRHWIEIVLLYRTYQSKDQTLGDKKDFDAFVSYAKWSSFPSEATSSLSEEHLALSLFPDVLENKYGYSLCLLERDVAPGGVYAEDIVSIIKRSRRGIFILSPNYVNGPSIFELQAAVNLALDDQTLKLILIKFCYF

SEQ ID NO: 52 - protein, 160 aa, Synthetic construct
SALLYRHWIEIVLLYRTYQSKDQTLGDKKDFDAFVSYAKWSSFPSEATSSLSEEHLALSLFPDVLENKYGYSLCLLERDVAPGGVYAEDIVSIIKRSRRGIFILSPNYVNGPSIFELQAAVNLALDDQTLKLILIKFCYFQEPESLPHLVKKALRVLPTV

SEQ ID NO: 53 - protein, 200 aa, Synthetic construct
SALLYRHWIEIVLLYRTYQSKDQTLGDKKDFDAFVSYAKWSSFPSEATSSLSEEHLALSLFPDVLENKYGYSLCLLERDVAPGGVYAEDIVSIIKRSRRGIFILSPNYVNGPSIFELQAAVNLALDDQTLKLILIKFCYFQEPESLPHLVKKALRVLPTVTWRGLKSVPPNSRFWAKMRYHMPVKNSQGFTWNQLRITSR

SEQ ID NO: 54 - protein, 29 aa, Synthetic construct
QLEKELQALEKENAQLEWELQALEKELAQ

SEQ ID NO: 55 - protein, 29 aa, Synthetic construct
QLEKKLQALKKKNAQLKWKLQALKKKLAQ

SEQ ID NO: 56 - protein, 44 aa, Synthetic construct
SHIQIPPGLTELLQGYTVEVLRQQPPDLVEFAVEYFTRLREARA

SEQ ID NO: 57 - protein, 18 aa, Synthetic construct
VQIEYLAKQIVDNAIQQA

SEQ ID NO: 58 - protein, 51 aa, Synthetic construct
IETRHSEIIKLENSIRELHDMFMDMAMLVESQGEMIDRIEYNVEHAVDYVE

SEQ ID NO: 59 - protein, 67 aa, Synthetic construct
ALSEIETRHSEIIKLENSIRELHDMFMDMAMLVESQGEMIDRIEYNVEHAVDYVERAVSDTKKAVKY

SEQ ID NO: 60 - protein, 69 aa, Synthetic construct
ELEEMQRRADQLADESLESTRRMLQLVEESKDAGIRTLVMLDEQGEQLERIEEGMDQINKDMKEAEKNL

SEQ ID NO: 61 - protein, 51 aa, Synthetic construct; identical to SEQ ID NO: 58

SEQ ID NO: 62 - protein, 180 aa, synthetic construct
SALLYRHWIEIVLLYRTYQSKDQTLGDKKDFDAFVSYAKWSSFPSEATSSLSEEHLALSLFPDVLENKYGYSLCLLERDVAPGGVYAEDIVSIIKRSRRGIFILSPNYVNGPSIFELQAAVNLALDDQTLKLILIKFCYFQEPESLPHLVKKALRVLPTVTWRGLKSVPPNSRFWAKMRY